(19) United States
(12) Patent Application Publication
(10) Pub. No.: US 2011/0009399 A1
(43) Pub. Date: Jan. 13, 2011

(54) ORGANIC COMPOUNDS

(76) Inventors: Peter Herold, Munchenstein (CH); Stjepan Jelakovic, Freiburg (DE); Robert Mah, Basel (CH); Dirk Behnke, Basel (CH); Stefan Stutz, Basel (CH); Vincenzo Tschinke, Binningen (CH); Nathalie Jotterand, Aclens (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/745,960

(22) PCT Filed: Dec. 12, 2008
(86) PCT No.: PCT/EP2008/067407

§ 371 Date: Sep. 21, 2010

(30) Foreign Application Priority Data
Dec. 13, 2007 (EP) 07150019.3

(51) Int. Cl. A61K 31/538; C07D 491/107; A61K 31/438; A61P 27/06; A61P 9/10; A61P 13/12; A61P 9/00; A61P 9/12

(52) U.S. Cl. 514/230.5; 546/17; 544/70; 514/278

(57) ABSTRACT

The application relates to trisubstituted piperidines of the general formula (I) and their salts, preferably their pharmaceutically acceptable salts, in which R¹, R^{2′}, X, U, W, m and n have the meanings explained in the description, a process for their preparation and the use of these compounds as medicines, especially as renin inhibitors.

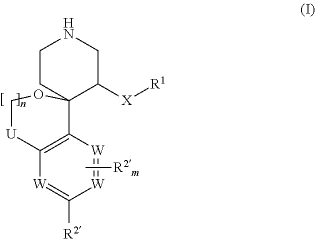

### FIELD OF THE INVENTION

The present invention relates to novel trisubstituted piperidines, processes for their preparation and the use of the compounds as medicines, especially as renin inhibitors.

### BACKGROUND OF THE INVENTION

Piperidine derivatives for use as medicines are disclosed for example in WO 97/09311. However, especially with regard to renin inhibition, there is still a need for highly potent active ingredients. In this context, the improvement of a compound's pharmacokinetic properties, resulting in better oral bioavailability, and/or it's overall safety profile are at the forefront. Properties directed towards better bioavailability are, for example, increased absorption, metabolic stability or solubility, or optimized lipophilicity. Properties directed towards a better safety profile are, for example, increased selectivity against drug metabolizing enzymes such as the cytochrome P450 enzymes.

### DETAILED DESCRIPTION OF THE INVENTION

The invention therefore relates firstly to trisubstituted piperidines of the general formula

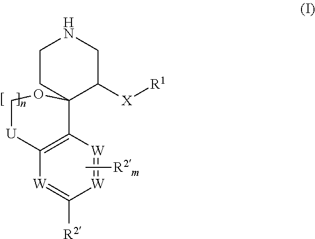

in which
R¹ is aryl or heterocyclyl, each of which is substituted by 1-4 radicals independently selected from the group consisting of
acyl-C₁₋₈-alkoxy-C₁₋₈-alkoxy,
acyl-C₁₋₈-alkoxy-C₁₋₈-alkyl,
(N-acyl)-C₁₋₈-alkoxy-C₁₋₈-alkylamino,
C₁₋₈-alkanoyl,
C₁₋₈-alkoxy,
C₁₋₈-alkoxy-C₁₋₈-alkanoyl,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
(N—C₁₋₈-alkoxy)-C₁₋₈-alkylaminocarbonyl-C₁₋₈-alkoxy,
(N—C₁₋₈-alkoxy)-C₁₋₈-alkylaminocarbonyl-C₁₋₈-alkyl,
C₁₋₈-alkoxy-C₁₋₈-alkylcarbamoyl,
C₁₋₈-alkoxy-C₁₋₈-alkylcarbonyl,
C₁₋₈-alkoxy-C₁₋₈-alkylcarbonylamino,
C₁₋₈-alkoxycarbonyl,
C₁₋₈-alkoxycarbonyl-C₁₋₈-alkoxy,
C₁₋₈-alkoxycarbonyl-C₁₋₈-alkyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₁₋₈-alkoxy-C₁₋₈-alkylcarbamoyl,
(N—C₁₋₈-alkyl)-C₁₋₈-alkoxy-C₁₋₈-alkylcarbonylamino,
(N—C₁₋₈-alkyl)-C₁₋₈-alkoxycarbonylamino,
(N—C₁₋₈-alkyl)-C₁₋₈-alkylcarbonylamino-C₁₋₈-alkoxy,
(N—C₁₋₈-alkyl)-C₁₋₈-alkylcarbonylamino-C₁₋₈-alkyl,
(N—C₁₋₈-alkyl)-C₁₋₈-alkylsulfonylamino-C₁₋₈-alkoxy,
(N—C₁₋₈-alkyl)-C₁₋₈-alkylsulfonylamino-C₁₋₈-alkyl,
C₁₋₈-alkylamidinyl,
C₁₋₈-alkylamino-C₁₋₈-alkoxy,
di-C₁₋₈-alkylamino-C₁₋₈-alkoxy,
C₁₋₈-alkylamino-C₁₋₈-alkyl,
di-C₁₋₈-alkylamino-C₁₋₈-alkyl,
C₁₋₈-alkylaminocarbonyl-C₁₋₈-alkoxy,
di-C₁₋₈-alkylaminocarbonyl-C₁₋₈-alkoxy,
C₁₋₈-alkylaminocarbonyl-C₁₋₈-alkoxy-C₁₋₈-alkyl,
C₁₋₈-alkylaminocarbonyl-C₁₋₈-alkyl,
di-C₁₋₈-alkylaminocarbonyl-C₁₋₈-alkyl,
C₁₋₈-alkylaminocarbonylamino-C₁₋₈-alkoxy,
C₁₋₈-alkylaminocarbonylamino-C₁₋₈-alkyl,
C₀₋₈-alkylcarbonylamino,
C₀₋₈-alkylcarbonylamino-C₁₋₈-alkoxy,
C₀₋₈-alkylcarbonylamino-C₁₋₈-alkyl,
C₁₋₈-alkylcarbonyloxy-C₁₋₈-alkoxy,
C₁₋₈-alkylcarbonyloxy-C₁₋₈-alkyl,
C₁₋₈-alkylsulfonyl,
C₁₋₈-alkylsulfonyl-C₁₋₈-alkoxy,
C₁₋₈-alkylsulfonyl-C₁₋₈-alkyl,
C₁₋₈-alkylsulfonylamino-C₁₋₈-alkoxy,
C₁₋₈-alkylsulfonylamino-C₁₋₈-alkyl,
optionally N-mono- or N,N-di-C₁₋₈-alkylated amino,
unsubstituted or substituted aryl-C₀₋₈-alkoxy,
unsubstituted or substituted aryl-C₀₋₈-alkyl, preferably halogen substituted-aryl,
optionally N-mono- or N,N-di-C₁₋₈-alkylated carbamoyl-C₀₋₈-alkoxy,
optionally N-mono- or N,N-di-C₁₋₈-alkylated carbamoyl-C₀₋₈-alkyl,
carboxy-C₁₋₈-alkoxy,
carboxy-C₁₋₈-alkoxy-C₁₋₈-alkyl,
carboxy-C₁₋₈-alkyl,
cyano,
cyano-C₁₋₈-alkoxy,
cyano-C₁₋₈-alkyl,
unsubstituted or substituted C₃₋₁₂-cycloalkyl-C₁₋₈-alkoxy,
unsubstituted or substituted C₃₋₁₂-cycloalkyl-C₁₋₈-alkyl,
unsubstituted or substituted C₃₋₁₂-cycloalkylcarbonylamino-C₁₋₈-alkoxy,
unsubstituted or substituted C₃₋₁₂-cycloalkylcarbonylamino-C₁₋₈-alkyl,
O,N-dimethylhydroxylamino-C₁₋₈-alkyl,
halogen,
halogen substituted C₁₋₈-alkoxy,
halogen substituted C₁₋₈-alkyl,
unsubstituted or substituted heterocyclyl-C₀₋₈-alkoxy,
unsubstituted or substituted heterocyclyl-C₀₋₈-alkyl, preferably C₁₋₈-alkoxy-C₁₋₈-alkylheterocyclyl,
unsubstituted or substituted heterocyclylcarbonyl,
hydroxy-C₁₋₈-alkoxy-C₁₋₈-alkoxy,
hydroxy-C₁₋₈-alkoxy-C₁₋₈-alkyl,
hydroxy-C₁₋₈-alkyl,
O-methyloximyl-C₁₋₈-alkyl,
oxide and oxo;
where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;
R^{2′} is independently selected from the group consisting of
C₁₋₈-alkanoyloxy-C₁₋₈-alkyl,
C₂₋₈-alkenyl,
C₂₋₈-alkenyloxy,
C₂₋₈-alkenyloxy-C₁₋₈-alkyl,
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
C₁₋₈-alkoxy-C₁₋₈-alkyl,
C₁₋₈-alkoxy-C₁₋₈-alkylamino-C₁₋₈-alkyl,
C₁₋₈-alkoxy-C₁₋₈-alkylsulfanyl,
C₁₋₈-alkoxy-C₁₋₈-alkylsulfanyl-C₁₋₈-alkyl,
C₁₋₈-alkoxycarbonyl,
C₁₋₈-alkoxycarbonyloxy-C₁₋₈-alkyl,
C₁₋₈-alkoxy-C₃₋₈-cycloalkyl-C₁₋₈-alkyl,
C₁₋₈-alkyl,
C₁₋₈-alkylsulfanyl,
C₁₋₈-alkylsulfanyl-C₁₋₈-alkoxy,
C₁₋₈-alkylsulfanyl-C₁₋₈-alkoxy-C₁₋₈-alkyl,
C₁₋₈-alkylsulfanyl-C₁₋₈-alkyl,
C₁₋₈-alkylsulfonyl-C₁₋₈-alkoxy-C₁₋₈-alkyl,
C₁₋₈-alkylsulfonyl-C₁₋₈-alkyl,
C₂₋₈-alkynyl,
optionally substituted C₁₋₈-alkoxy
optionally N-mono- or N,N-di-C₁₋₈-alkylated amino-C₁₋₈-alkoxy,
optionally N-mono- or N,N-di-C₁₋₈-alkylated amino-carbonyl-C₁₋₈-alkyl,
unsubstituted or substituted aryl-C₁₋₈-alkoxy-C₁₋₈-alkoxy,
unsubstituted or substituted aryl-heterocyclyl-C₀₋₈-alkoxy,
unsubstituted or substituted heterocyclyl-heterocyclyl-C₀₋₈-alkoxy,
unsubstituted or substituted aryloxy,
unsubstituted or substituted aryl-C₀₋₈-alkoxy-C₁₋₈-alkoxy,
unsubstituted or substituted aryl-C₀₋₈-alkoxy-C₁₋₈-alkoxy-C₁₋₈-alkyl,
carboxy-C₁₋₈-alkyl,
cyano,
cyano-C₁₋₈-alkyl,
unsubstituted or substituted C₃₋₈-cycloalkyl-C₀₋₈-alkoxy-C₁₋₈-alkoxy,
unsubstituted or substituted C₃₋₈-cycloalkyl-C₀₋₈-alkoxy-C₁₋₈-alkoxy-C₁₋₈-alkyl,
unsubstituted or substituted C₃₋₈-cycloalkyl-C₀₋₈-alkoxy-C₁₋₈-alkyl, preferably C₁₋₈-alkoxy-C₀₋₈-alkyl-C₃₋₈-cycloalkyl-C₀₋₈-alkoxy-C₁₋₈-alkyl,
unsubstituted or substituted C₃₋₈-cycloalkyl-C₀₋₈-alkylamino-C₁₋₈-alkyl,
halogen-substituted C₁₋₈-alkoxy,
halogen-substituted C₁₋₈-alkyl,
halogen-substituted C₁₋₈-alkoxy-C₁₋₈-alkoxy-C₁₋₈-alkyl,
unsubstituted or substituted heterocyclyl-carbonyl-C₁₋₈-alkyl,
unsubstituted or substituted heterocyclyl-C₁₋₈-alkyl,
unsubstituted or substituted heterocyclyl-sulfanyl-C₁₋₈-alkoxy-C₁₋₈-alkyl,
unsubstituted or substituted heterocyclyl-C₀₋₈-alkoxy-C₁₋₈-alkoxy and
unsubstituted or substituted heterocyclyl-C₀₋₈-alkoxy-C₁₋₈-alkyl;
X is -Alk-, —O-Alk-, -Alk-O—, —O-Alk-O—, —S-Alk-, -Alk-S—, -Alk-NR⁴—, —NR⁴-Alk-, —C(O)—NR⁴—, -Alk-C(O)—NR⁴—, -Alk-C(O)—NR⁴-Alk-, —NR⁴—C(O)—, -Alk-NR⁴—C(O)—, —NR⁴—C(O)-Alk-, -Alk-NR⁴—C(O)-Alk-, —O-Alk-C(O)—NR⁴—, —O-Alk-NR⁴—C(O)—, —S(O)₂—NR⁴— or —S(O)₂—NR⁴-Alk-, where Alk is C₁₋₈-alkylene which may optionally be substituted by halogen;
   R⁴ is hydrogen, C₁₋₈-alkyl, C₁₋₈-alkoxy-C₁₋₈-alkyl, acyl, unsubstituted or substituted C₃₋₈-cycloalkyl or unsubstituted or substituted aryl-C₁₋₈-alkyl;
   U is selected from the group consisting of —CH₂—, NR⁴, —O— and S(O)ₚ;
   W is independently selected from the group consisting of —CH═ and —N═, whereby a maximum of one W can be —N═;

### n is 0-2 if U is —CH₂— or n is 2 if U is —O—, NR⁴, or S(O)ₚ;

### m is 0-3 if all W are —CH═; or m is 0-2, if one W is —N═; and

p is 0-2
and the salts thereof, preferably the pharmaceutically acceptable salts thereof.

The linkage of the above (and hereinafter) mentioned substituent —X— within the compound of the formula (I) starts from the piperidine ring with the substituent —X— being arranged from left to right when written as indicated above. For example, the fragment “—X—R¹” of the compound of the formula (I) with X meaning “—NR⁴-Alk-” is: “—NR⁴-Alk-R¹”.

Ranges for the number of radicals referred to as, for example, “n is 0-2” include the numbers given as the endpoints of the range and any integer in the range; thus n may take the value of zero, one or two.

The meaning of “C₀-alkyl” in the above (and hereinafter) mentioned C₀₋₈-alkyl groups is a bond or, if located at a terminal position, a hydrogen atom.

The meaning of “C₀-alkoxy” in the above (and hereinafter) mentioned C₀₋₈-alkoxy groups is “—O—” or, if located at a terminal position, an —OH group.

C₁₋₈-Alkyl and alkoxy radicals may be linear or branched. Examples of C₁₋₈-alkyl and alkoxy radicals are methyl, ethyl, n-propyl, isopropyl, n-butyl, isobutyl, sec-butyl, tertbutyl, pentyl, hexyl, and methoxy, ethoxy, propoxy, isopropoxy, butoxy, isobutoxy, sec-butoxy and tert-butoxy. C₁₋₈-Alkylenedioxy radicals are preferably methylene-dioxy, ethylenedioxy and propylenedioxy. C₁₋₈-Alkanoyl refers to C₁₋₈-alkylcarbonyl. Examples of C₁₋₈-alkanoyl radicals are acetyl, propionyl and butyryl.

As part of the substituent on R¹,
cycloalkyl refers to a saturated, cyclic hydrocarbon radical having 3 to 12 carbon atoms, for example cyclopropyl, cyclobutyl, cyclopentyl, cyclohexyl, cycloheptyl, bicyclo[2.2.1]heptyl, cyclooctyl, bicyclo[2.2.2]octyl and adamantyl, and may be unsubstituted or substituted one or more times, e.g. substituted once or twice by C₁₋₈-alkanoyl, C₂₋₈-alkenyl, C₂₋₈-alkynyl, C₁₋₈-alkoxy, C₁₋₈-alkoxy-C₁₋₈-alkoxy, C₁₋₈-alkoxy-C₁₋₈-alkyl, C₁₋₈-alkoxycarbonylamino, C₁₋₈-alkyl, C₀₋₈-alkylcarbonylamino, C₁₋₈-alkylcarbonyloxy, C₁₋₈-alkylenedioxy, optionally N-mono- or N,N-di-C₁₋₈-alkylated amino, aryl, optionally N-mono- or N,N-di-C₁₋₈-alkylated carbamoyl, optionally esterified carboxy, cyano, C₃₋₈-cyclo-alkoxy, halogen, heterocyclyl, hydroxy, oxo, halogen-substituted C₁₋₈-alkoxy or halogen-substituted C₁₋₈-alkyl.

As part of the substituent R^{2′} or as R⁴,
cycloalkyl refers to a saturated cyclic hydrocarbon radicals having 3 to 8 carbon atoms, for example cyclopropyl, cyclobutyl or cyclopentyl and may be unsubstituted or substituted once or twice by C₁₋₈-alkoxy, C₁₋₈-alkoxy-C₁₋₈-alkyl, optionally halogen substituted C₁₋₈-alkyl or halogen.

Cycloalkyl radicals with two connection points may be linked via 2 different carbon atoms or via the same carbon atom, for example 1,1-cyclopropyl or 1,2-cyclopropyl.

C₁₋₈-Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₈-alkenylene radicals are, for example, vinylene and propenylene; C₂₋₈-alkynylene radicals are, for example, ethynylene; acyl radicals are alkanoyl radicals, preferably C₁₋₈-alkanoyl radicals, or aroyl radicals such as benzoyl.

As R¹,
aryl refers to mono- or polynuclear aromatic radicals which may be substituted one or more times, e.g. substituted once or twice, such as, for example, phenyl, substituted phenyl, naphthyl, substituted naphthyl. Aryl refers also to bicyclic systems, where a monocyclic aryl radical has a 3-7-membered fused-on carbocyclic ring, such as, for example tetrahydronaphthyl or substituted tetrahydronaphthyl.

As part of a substituent on R¹, or as part of the substituent R^{2′} or R⁴,
aryl refers to mononuclear aromatic radicals which may be substituted one or more times, e.g. substituted once or twice by C₁₋₈-alkoxy, C₁₋₈-alkyl, optionally esterified carboxy, cyano, halogen, hydroxy, halogen substituted C₁₋₈-alkoxy, halogen substituted C₁₋₈-alkyl or phenyl, such as, for example, phenyl or substituted phenyl.

For R¹,
the term heterocyclyl refers to 3-16-membered, mono-, bi- or polycyclic, saturated, unsaturated and partially unsaturated heterocyclic radicals having 1 to 4 nitrogen and/or 1 or 2 sulfur or oxygen atoms. Preference is given to 3-8-membered, particularly preferably 5- or 6-membered, monocyclic radicals which optionally have a 3-8-membered fused-on ring, which may be carbocyclic or heterocyclic. A further preferred group of heterocyclic radicals are bi- or polycyclic heterocycles which optionally have a spirocyclic or bridged ring. Preferred heterocyclic radicals have in each ring 1 nitrogen, oxygen or sulfur atom, 1-2 nitrogen atoms and 1-2 oxygen atoms or 1-2 nitrogen atoms and 1-2 sulfur atoms, with at least one, preferably 1-7, carbon atoms being present in each ring. Heterocyclic radicals may be substituted one or more times, in particular once, twice or three times.
Examples of unsaturated heterocyclyl radicals are
benzo[1,3]dioxolyl,
benzofuranyl,
benzoimidazolyl,
benzooxazolyl,
benzothiazolyl,
benzo[b]thienyl,
quinazolinyl,
quinolyl,
quinoxalinyl,
2H-chromenyl,
dihydrobenzofuranyl,
1,3-dihydrobenzoimidazolyl,
3,4-dihydro-2H-benzo[1,4]oxazinyl,
3,4-dihydro-3H-benzo[1,4]oxazinyl,
1,4-dihydrobenzo[d][1,3]oxazinyl,
3,4-dihydro-2H-benzo[1,4]thiazinyl,
3,4-dihydro-1H-quinazolinyl,
3,4-dihydro-1H-quinolinyl,
2,3-dihydroindolyl,
2,3-dihydro-1H-pyrido[2,3-b][1,4]oxazinyl,
1,1-dioxodihydro-2H-benzo[1,4]thiazinyl,
furyl,
imidazolyl,
imidazo[1,5-a]pyridinyl,
imidazo[1,2-a]pyrimidinyl,
indazolyl,
indolyl,
isobenzofuranyl,
isoquinolyl,
[1,5]naphthyridyl,
oxazolyl,
phthalazinyl,
pyranyl,
pyrazinyl,
pyrazolyl,
pyridyl,
pyrimidinyl,
1H-pyrrolizinyl,
pyrrolo[3,2-c]pyridinyl,
pyrrolo[2,3-c]pyridinyl,
pyrrolo[3,2-b]pyridinyl,
1H-pyrrolo[2,3-b]pyridyl,
pyrrolyl,
1,3,4,5-tetrahydrobenzo[b]azepinyl,
tetrahydroquinolinyl,
tetrahydroquinoxalinyl,
tetrahydroisoquinolinyl,
thiazolyl,
thienyl,
triazinyl and
triazolyl.
   Examples of saturated heterocyclyl radicals are
azepanyl,
azetidinyl,
aziridinyl,
3,4-dihydroxypyrrolidinyl,
2,6-dimethylmorpholinyl,
3,5-dimethylmorpholinyl,
dioxanyl,
[1,4]dioxepanyl,
dioxolanyl,
4,4-di-oxothiomorpholinyl,
dithianyl,
dithiolanyl,
2-hydroxymethylpyrrolidinyl,
4-hydroxypiperidinyl,
3-hydroxypyrrolidinyl,
4-methylpiperazinyl,
1-methylpiperidinyl,
1-methylpyrrolidinyl,
morpholinyl,
oxathianyl,
oxepanyl,
piperazinyl,
piperidinyl,
pyrrolidinyl,
tetrahydrofuranyl,
tetrahydropyranyl,
tetrahydrothiophenyl,
tetrahydrothiopyranyl,
thiepanyl and
thiomorpholinyl.
   Examples of bi- or polycyclic saturated or partially unsaturated heterocyclyl radicals are
2,5-dioxabicyclo[4.1.0]heptanyl,
2-oxa-bicyclo[2.2.1]heptanyl,
2-oxabicyclo[4.1.0]heptanyl,
3-oxabicyclo[4.1.0]heptanyl,
7-oxa-bicyclo[2.2.1]heptanyl,
2-oxabicyclo[3.1.0]hexanyl,
3-oxabicyclo[3.1.0]hexanyl,
1-oxa-spiro[2.5]octanyl,
6-oxaspiro[2.5]octanyl, 3-oxabicyclo[3.3.1]nonanyl,
1a,7b-dihydro-1H-cyclopropa[c]chromenyl and
1,1a,2,7b-tetrahydrocyclopropa[c]chromenyl.

As part of a substituent on R¹,
the term heterocyclyl refers to 3-7 membered monocyclic, saturated and unsaturated heterocyclic radicals having 1 to 4 nitrogen and/or 1 or 2 sulfur or oxygen atoms, which may be substituted one or more times, such as, for example, substituted once or twice by C₁₋₈-alkoxy, C₁₋₈-alkyl, C₁₋₈-alkoxy-C₁₋₈-alkyl, optionally esterified carboxy, cyano, halogen, hydroxy, halogen-substituted C₁₋₈-alkoxy or halogen-substituted C₁₋₈-alkyl.
Examples of such heterocyclyl radicals are
imidazolyl,
morpholinyl,
oxetanyl,
oxiranyl,
pyrazolyl,
pyridyl,
pyrrolidinyl,
tetrahydrofuranyl,
tetrahydropyranyl,
tetrazolyl,
thiazolyl and
triazolyl.

As part of the substituent R^{2′},
the term heterocyclyl refers to 3-7 membered monocyclic, saturated, partially unsaturated and maximally unsaturated heterocyclic radicals having 1 to 5 nitrogen and/or 1 or 2 sulfur or oxygen atoms, which may be substituted one or more times, such as, for example, substituted once, twice or three times by C₁₋₈-alkoxy, C₁₋₈-alkoxy-C₁₋₈-alkyl, C₁₋₈-alkyl, aryl, cyano, halogen, heterocyclyl, hydroxy, halogen substituted C₁₋₈-alkoxy or halogen substituted C₁₋₈-alkyl.
Examples of such heterocycles are
imidazolyl,
oxetanyl,
pyrazolyl.
pyrrolidinyl,
tetrazolyl,
thiazolyl and
triazolyl.

Heterocyclyl radicals which comprise a nitrogen atom may be linked either via the N atom or via a C atom to the remainder of the molecule.

Hydroxy-substituted C₁₋₈-alkoxy may be for example hydroxy-C₁₋₈-alkoxy or else polyhydroxy-C₁₋₈-alkoxy.

The term halogen-substituted C₁₋₈-alkyl refers to C₁₋₈-alkyl radicals which may be substituted by 1-8 halogen atoms, such as, for example, bromo, chloro, fluoro, iodo. An analogous statement applies to radicals, such as halogen-substituted C₁₋₈-alkoxy.

In the context of this invention whenever a substitution is described as occurring more than once, said substitution, for example twice, consists of substituents independently selected from the list of substituents given and thus is either two different substituents or twice the same substituent.

The compounds of the formula (I) have at least two asymmetric carbon atoms and may therefore exist in the form of optically pure diastereomers, diastereomeric mixtures, diastereomeric racemates, mixtures of diastereomeric racemates or as meso compounds. The invention encompasses all these forms. Mixtures of diastereomers, diastereomeric racemates or mixtures of diastereomeric racemates can be fractionated by conventional methods, e.g. by column chromatography, thin-layer chromatography, HPLC and the like.

Salts are primarily the pharmaceutically acceptable or nontoxic salts of compounds of formula (I). The term “pharmaceutically acceptable salts” encompasses salts with inorganic or organic acids, such as hydrochloric acid, hydrobromic acid, nitric acid, sulfuric acid, phosphoric acid, citric acid, formic acid, maleic acid, acetic acid, succinic acid, tartaric acid, methanesulfonic acid, p-toluenesulfonic acid and the like.

Salts of compounds having salt-forming groups are in particular acid addition salts, salts with bases, or, in the presence of a plurality of salt-forming groups, in some cases also mixed salts or internal salts.

Such salts are formed, for example, from compounds of formula (I) with an acidic group, for example a carboxyl or sulfonyl group, and are, for example, the salts thereof with suitable bases such as non-toxic metal salts derived from metals of group Ia, Ib, IIa and IIb of the Periodic Table of the Elements, for example alkali metal, in particular lithium, sodium, or potassium, salts, alkaline earth metal salts, for example magnesium or calcium salts, and also zinc salts and ammonium salts, including those salts which are formed with organic amines, such as optionally hydroxy-substituted mono-, di- or trialkylamines, in particular mono-, di- or tri(lower alkyl)amines, or with quaternary ammonium bases, e.g. methyl-, ethyl-, diethyl- or triethylamine, mono-, bis- or tris(2-hydroxy(lower alkyl))amines, such as ethanol-, diethanol- or triethanolamine, tris(hydroxymethyl)methylamine or 2-hydroxy-tert-butylamine, N,N-di(lower alkyl)-N-(hydroxy(lower alkyl))amine, such as N,N-di-N-dimethyl-N-(2-hydroxyethyl)amine, or N-methyl-D-glucamine, or quaternary ammonium hydroxides such as tetrabutyl ammoniumhydroxide. The compounds of formula (I) having a basic group, for example an amino group, may form acid addition salts, for example with suitable inorganic acids, e.g. hydrohalic acid such as hydrochloric acid, hydrobromic acid, sulfuric acid with replacement of one or both protons, phosphoric acid with replacement of one or more protons, e.g. ortho-phosphoric acid or metaphosphoric acid, or pyrophosphoric acid with replacement of one or more protons, or with organic carboxylic, sulfonic or phosphonic acids or N-substituted sulfamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, and also amino acids, for example the alpha-amino acids mentioned above, and also methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, 2- or 3-phosphoglycerate, glucose 6-phosphate, N-cyclohexylsulfamic acid (with formation of the cyclamates) or with other acidic organic compounds such as ascorbic acid. Compounds of formula (I) having acidic and basic groups may also form internal salts.

Salts obtained may be converted to other salts in a manner known per se, acid addition salts, for example, by treating with a suitable metal salt such as a sodium, barium or silver salt, of another acid in a suitable solvent in which an inorganic salt which forms is insoluble and thus separates out of the reaction equilibrium, and base salts by release of the free acid and salt reformation.

The compounds of formula (I), including their salts, may also be obtained in the form of hydrates or include the solvent used for the crystallization.

For the isolation and purification, pharmaceutically unsuitable salts may also find use.

The compound groups mentioned throughout the description are not to be regarded as closed, but rather parts of these compound groups may be exchanged with one another or with the definitions given above or omitted in a sensible manner, for example to replace general by more specific definitions. The definitions are valid in accordance with general chemical principles, such as, for example, the common valences for atoms.

Preferred compounds according to the invention are those of the general formula (IA) and the salts thereof, preferably the pharmaceutically acceptable salts thereof.

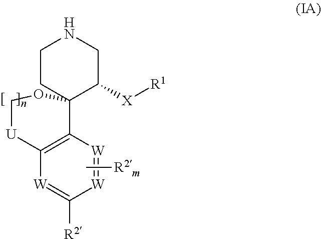

in which R¹, R^{2′}, X, U, W, m and n have the meaning indicated above for the compounds of the formula (I).

A further preferred group of compounds of the formula (I), and particularly preferably of the formula (IA), and the salts thereof, preferably the pharmaceutically acceptable salts thereof, are compounds in which

W is in each case —CH═.

A further preferred group of compounds of the formula (I), and particularly preferably of the formula (IA), and the salts thereof, preferably the pharmaceutically acceptable salts thereof, are compounds in which

W is independently selected from —CH═ or —N═, with exactly one W being —N═.

A further preferred group of compounds of the formula (I), and particularly preferably of the formula (IA), and the salts thereof, preferably the pharmaceutically acceptable salts thereof, are compounds in which

R¹ is phenyl or heterocyclyl, each substituted as indicated above for compounds of the formula (I).

A further preferred group of compounds of the formula (I), and particularly preferably of the formula (IA), and the salts thereof, preferably the pharmaceutically acceptable salts thereof, are compounds in which

U is —CH₂— and n is 0-2 and in which R¹, R^{2′}, W, X and m have the meaning indicated above for the compounds of the formula (I).

A further preferred group of compounds of the formula (I), and particularly preferably of the formula (IA), and the salts thereof, preferably the pharmaceutically acceptable salts thereof, are compounds in which

U is —O— and n is 2 and in which R¹, R^{2′}, W, X and m have the meaning indicated above for the compounds of the formula (I).

Particularly preferred heterocyclic radicals R¹ are
benzo[1,3]dioxolyl,
benzofuranyl,
benzoimidazolyl,
4H-benzo[1,4]oxazinyl,
benzooxazolyl,
4H-benzo[1,4]thiazinyl,
quinolinyl,
2H-chromenyl,
dihydro-benzo[e][1,4]diazepinyl,
3,4-dihydro-2H-benzo[1,4]oxazinyl,
3,4-dihydro-3H-benzo[1,4]oxazinyl,
1,4-dihydro-2H-benzo[d][1,3]oxazinyl,
3,4-dihydro-2H-benzo[1,4]thiazinyl,
1a,7b-dihydro-1H-cyclopropa[c]chromenyl,
1,3-dihydroindolyl,
2,3-dihydroindolyl,
2,3-dihydro-1H-pyrido[2,3-b][1,4]oxazinyl,
imidazo[1,5-a]pyridinyl,
indazolyl,
indolyl,
3H-isobenzofuranyl,
[1,5]naphthyridyl,
oxazolyl,
phthalazinyl,
pyrazolyl,
1H-pyrido[2,3-b][1,4]oxazinyl,
pyridyl,
pyrimidinyl
1H-pyrrolizinyl,
1H-pyrrolo[2,3-b]pyridyl,
pyrrolyl,
tetrahydrobenzo[e][1,4]diazepinyl,
2H-thieno[2,3-d]pyrimidinyl,
tetrahydro-quinoxalinyl,
1,1a,2,7b-tetrahydrocyclopropa[c]chromenyl and
triazinyl.

Particularly preferred radicals R¹ are
benzo[1,3]dioxolyl,
benzofuranyl,
benzoimidazolyl,
4H-benzo[1,4]oxazinyl,
benzooxazolyl,
4H-benzo[1,4]thiazinyl,
2H-chromenyl,
dihydro-benzo[e][1,4]diazepinyl,
3,4-dihydro-2H-benzo[1,4]oxazinyl,
3,4-dihydro-3H-benzo[1,4]oxazinyl,
1,4-dihydro-2H-benzo[d][1,3]oxazinyl,
3,4-dihydro-2H-benzo[1,4]thiazinyl,
1a,7b-dihydro-1H-cyclopropa[c]chromenyl,
1,3-dihydroindolyl,
2,3-dihydroindolyl,
2,3-dihydro-1H-pyrido[2,3-b][1,4]oxazinyl,
imidazo[1,5-a]pyridinyl,
indazolyl,
indolyl,
3H-isobenzofuranyl,
1H-pyrido[2,3-b][1,4]oxazinyl,
phenyl,
pyridyl,
pyrimidinyl
1H-pyrrolo[2,3-b]pyridyl,
1,1a,2,7b-tetrahydrocyclopropa[c]chromenyl and
triazinyl;
   substituted by 1-3 radicals independently selected from the group consisting of
C₁₋₈-alkanoyl,
C₁₋₈-alkoxy,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
(N—C₁₋₈-alkoxy)-C₁₋₈-alkylaminocarbonyl-C₁₋₈-alkoxy,
(N—C₁₋₈-alkoxy)-C₁₋₈-alkylaminocarbonyl-C₁₋₈-alkyl,
C₁₋₈-alkoxy-C₁₋₈-alkylcarbonyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₀₋₈-alkylcarbonylamino-C₁₋₈-alkoxy,
(N—C₁₋₈-alkyl)-C₀₋₈-alkylcarbonylamino-C₁₋₈-alkyl,
C₀₋₈-alkylcarbonylamino-C₁₋₈-alkoxy,
C₀₋₈-alkylcarbonylamino-C₁₋₈-alkyl,
halogen,
oxide,
oxo,
halogen substituted C₁₋₈-alkoxy,
halogen substituted C₁₋₈-alkyl,
unsubstituted or substituted heterocyclyl-C₁₋₈-alkoxy and
unsubstituted or substituted heterocyclyl-C₁₋₈-alkyl.

R¹ is very particularly preferably
2H-chromenyl,
3,4-dihydro-2H-benzo[1,4]oxazinyl,
3,4-dihydro-2H-benzo[1,4]thiazinyl or
1,3-dihydroindolyl
   substituted by 1-3 radicals independently selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
C₁₋₈-alkoxy-C₁₋₈-alkylcarbonyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₀₋₈-alkylcarbonylamino-C₁₋₈-alkoxy,
(N—C₁₋₈-alkyl)-C₀₋₈-alkylcarbonylamino-C₁₋₈-alkyl,
C₀₋₈-alkylcarbonylamino-C₁₋₈-alkoxy,
C₀₋₈-alkylcarbonylamino-C₁₋₈-alkyl,
halogen,
oxo,
halogen-substituted C₁₋₈-alkoxy and
halogen-substituted C₁₋₈-alkyl.

Preference is furthermore given to compounds of the formulae (I) and (IA) and the salts thereof, preferably the pharmaceutically acceptable salts thereof, in which R^{2′} is independently selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
C₁₋₈-alkoxy-C₁₋₈-alkyl,
C₁₋₈-alkoxy-C₀₋₈-alkyl-C₃₋₈-cycloalkyl-C₀₋₈-alkoxy-C₁₋₈-alkyl,
C₁₋₈-alkoxy-C₁₋₈-alkylsulfanyl,
C₁₋₈-alkoxy-C₁₋₈-alkylsulfanyl-C₁₋₈-alkyl,
C₁₋₈-alkoxy-C₃₋₈-cycloalkyl-C₁₋₈-alkyl,
C₁₋₈-alkyl,
C₁₋₈-alkylsulfanyl-C₁₋₈-alkoxy,
C₁₋₈-alkylsulfanyl-C₁₋₈-alkoxy-C₁₋₈-alkyl,
optionally substituted C₁₋₈-alkoxy
unsubstituted or substituted aryl-heterocyclyl-C₀₋₈-alkoxy,
unsubstituted or substituted C₃₋₈-cycloalkyl-C₀₋₈-alkoxy-C₁₋₈-alkyl,
halogen-substituted C₁₋₈-alkoxy,
halogen-substituted C₁₋₈-alkyl,
unsubstituted or substituted heterocyclyl-C₀₋₈-alkoxy-C₁₋₈-alkyl,
unsubstituted or substituted heterocyclyl-heterocyclyl-C₀₋₈-alkoxy,
unsubstituted or substituted aryl-C₀₋₈-alkoxy-C₁₋₈-alkoxy and
unsubstituted or substituted aryl-C₀₋₈-alkoxy-C₁₋₈-alkoxy-C₁₋₈-alkyl;
   R^{2′} is particularly preferably selected from
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
optionally substituted C₁₋₈-alkoxy,
C₁₋₈-alkyl,
unsubstituted or substituted C₃₋₈-cycloalkyl-C₀₋₈-alkoxy-C₁₋₈-alkyl,
unsubstituted or substituted heterocyclyl-C₀₋₈-alkoxy-C₁₋₈-alkyl and
unsubstituted or substituted heterocyclyl-pyrrolidinyl-C₀₋₈-alkoxy;
   R^{2′} is very particularly preferably selected from
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkyl,
optionally substituted C₁₋₈-alkoxy,
C₁₋₈-alkyl,
unsubstituted or substituted C₃₋₈-cycloalkyl-C₀₋₈-alkoxy-C₁₋₈-alkyl,
unsubstituted or substituted heterocyclyl-C₀₋₈-alkoxy-C₁₋₈-alkyl and
unsubstituted or substituted heterocyclyl-pyrrolidinyl-C₀₋₈-alkoxy.

A further preferred group of compounds of the formula (I), and particularly preferably of the formula (IA), and the salts thereof, preferably the pharmaceutically usable salts thereof, are compounds in which

X is -Alk-, —O-Alk- or —O-Alk-O— where Alk is C₁₋₈-alkylene.
X is particularly preferred —O-Alk-, and very particularly preferred —O—CH₂—.

Very particular preference is given to compounds and the salts thereof, preferably the pharmaceutically acceptable salts thereof, of the formulae (I) and (IA) in which

R¹ is 2H-chromenyl or 3,4-dihydro-2H-benzo[1,4]oxazinyl, substituted as defined for compounds of formula (I);
R^{2′} is selected from
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkyl,
optionally substituted C₁₋₈-alkoxy,
C₁₋₈-alkyl,
unsubstituted or substituted C₃₋₈-cycloalkyl-C₀₋₈-alkoxy-C₁₋₈-alkyl,
unsubstituted or substituted heterocyclyl-C₀₋₈-alkoxy-C₁₋₈-alkyl and
unsubstituted or substituted heterocyclyl-pyrrolidinyl-C₀₋₈-alkoxy;
   X is -Alk-, —O-Alk- or —O-Alk-O— where Alk is C₁₋₈-alkylene;
   U is selected from the group consisting of —CH₂— and —O—;
   W is in each case —CH═;

### n is 0-2 if U is —CH₂— or n is 2 if U is —O—; and

m is 0.

The compounds of the formulae (I) and (IA) can be prepared in an analogous manner to preparation processes disclosed in the literature. Similar preparation processes are described for example in WO 97/09311 and WO 00/063173. Details of the specific preparation variants can be found in the examples.

The compounds of the formula (I) can also be prepared in optically pure form. Separation into antipodes can take place by methods known per se, either preferably at an early stage in the synthesis by salt formation with an optically active acid such as, for example, (+)- or (−)-mandelic acid and separation of the diastereomeric salts by fractional crystallization or preferably at a rather late stage by derivatizing with a chiral auxiliary component such as, for example, (+)- or (−)-camphanoyl chloride, and separation of the diastereomeric products by chromatography and/or crystallization and subsequent cleavage of the linkage to the chiral auxiliary. The pure diastereomeric salts and derivatives can be analysed to determine the absolute configuration of the contained piperidine by conventional spectroscopic methods, with X-ray spectroscopy on single crystals representing a particularly suitable method.

It is possible for the configuration at individual chiral centres in a compound of formula (I) to be inverted selectively. For example, the configuration of asymmetric carbon atoms which bear nucleophilic substituents, such as amino or hydroxyl, may be inverted by second-order nucleophilic substitution, if appropriate after conversion of the bonded nucleophilic substituent to a suitable nucleofugic leaving group and reaction with a reagent which introduces the original substituents, or the configuration at carbon atoms having hydroxyl groups can be inverted by oxidation and reduction, analogously to the process in the European patent application EP-A-0 236 734. Also advantageous is the reactive functional modification of the hydroxyl group and subsequent replacement thereof by hydroxyl with inversion of configuration.

The compounds of the formula (I) and (IA) also include compounds in which one or more atoms are replaced by their stable, non-radioactive isotopes; for example a hydrogen atom by deuterium.

The compounds of the formula (I) and (IA) also include compounds that have been nitrosated through one or more sites such as oxygen (hydroxyl condensation), sulphur (sulphydryl condensation) and/or nitrogen. The nitrosated compounds of the present invention can be prepared using conventional methods known to one skilled in the art. For example, known methods for nitrosating compounds are described in WO2004/098538 A2.

The compounds of the formula (I) and (IA) also include compounds that have been converted at one or more sites such that a nitrate-ester-containing linker is attached to an existing oxygen and/or nitrogen. Preferred derivatives are compounds where either the piperidine nitrogen atom or a sidechain nitrogen atom in R¹ of formula (I) has been converted to either an amide or carbamate group possessing a nitrate-ester-containing linker, for example>N—C(O)-L-ONO₂ or >NC(O)—O-L-ONO₂, where L represents a linker such as C₁₋₈-alkyl or aryl-C₁₋₈-alkyl. Further preferred derivatives are compounds where the oxygen atom of a hydroxyl group in R¹ of formula (I) has been converted to either an ester or carbonate group possessing a nitrate-ester-containing linker, for example —O—(C═O)-L-ONO₂ or —O—(C═O)—O-L-ONO₂, where L represents a linker such as C₁₋₈-alkyl or aryl-C₁₋₈-alkyl. Such “nitroderivatives” of the compounds of the present invention can be prepared using conventional methods known to one skilled in the art. For example, known methods for converting compounds into their nitroderivatives are described in WO 2007/045551 A2.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S— and O-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower omega-(amino, mono- or dialkylamino, carboxy, lower alkoxycarbonyl)—alkyl esters or such as lower α-(alkanoyloxy, alkoxycarbonyl or dialkylaminocarbonyl)—alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate.

The compounds of the formula (I), and preferably of the formula (IA), and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radioimmunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/l.

Illustrative of the invention, the compounds of examples 2, 7 and 14 inhibit the formation of angiotensin I with IC₅₀ values in the range of about 1-20·10⁻⁹ mol/l.

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulfonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The blood pressure-reducing effect of the compounds described herein can be tested in vivo using the following protocol:

The investigations take place in 5 to 6-week old, male double transgenic rats (dTGR), which overexpress both human angiotensinogen and human renin and consequently develop hypertension (Bohlender J. et al., J. Am. Soc. Nephrol. 2000; 11: 2056-2061). This double transgenic rat strain was produced by crossbreeding two transgenic strains, one for human angiotensinogen with the endogenous promoter and one for human renin with the endogenous promoter. Neither single transgenic strain was hypertensive. The double transgenic rats, both males and females, develop severe hypertension (mean systolic pressure, approximately 200 mm Hg) and die after a median of 55 days if untreated. The fact that human renin can be studied in the rat is a unique feature of this model. Age-matched Sprague-Dawley rats serve as non-hypertensive control animals. The animals are divided into treatment groups and receive test substance or vehicle (control) for various treatment durations. The applied doses for oral administration may range from 0.5 to 100 mg/kg body weight. Throughout the study, the animals receive standard feed and tap water ad libitum. The systolic and diastolic blood pressure, and the heart rate are measured telemetrically by means of transducers implanted in the abdominal aorta, allowing the animals free and unrestricted movement.

The effect of the compounds described herein on kidney damage (proteinuria) can be tested in vivo using the following protocol:

The investigations take place in 4-week old, male double transgenic rats (dTGR), as described above. The animals are divided into treatment groups and receive test substance or vehicle (control) each day for 7 weeks. The applied doses for oral administration may range from 0.5 to 100 mg/kg body weight. Throughout the study, the animals receive standard feed and tap water ad libitum. The animals are placed periodically in metabolism cages in order to determine the 24-hour urinary excretion of albumin, diuresis, natriuresis, and urine osmolality. At the end of the study, the animals are sacrificed and the kidneys and hearts may also be removed for determining the weight and for immunohistological investigations (fibrosis, macrophage/T cell infiltration, etc.).

The bioavailability of the compounds described herein can be tested in vivo using the following protocol:

The investigations take place in pre-catheterized (carotid artery) male rats (300 g±20%) that can move freely throughout the study. The compound is administered intravenously and orally (gavage) in separate sets of animals. The applied doses for oral administration may range from 0.5 to 50 mg/kg body weight; the doses for intravenous administration may range from 0.5 to 20 mg/kg body weight. Blood samples are collected through the catheter before compound administration and over the subsequent 24-hour period using an automated sampling device (AccuSampler, DiLab Europe, Lund, Sweden). Plasma levels of the compound are determined using a validated LC-MS analytical method. The pharmacokinetic analysis is performed on the plasma concentration-time curves after averaging all plasma concentrations across time points for each route of administration. Typical pharmacokinetics parameters to be calculated include: maximum concentration (Cₘₐₓ), time to maximum concentration (tₘₐₓ), area under the curve from 0 hours to the time point of the last quantifiable concentration (AUC₀₋ₜ), area under the curve from time 0 to infinity (AUC_{0-inf}), elimination rate constant (K), terminal half-life (t_{1/2}), absolute oral bio-availability or fraction absorbed (F), clearance (CL), and Volume of distribution during the terminal phase (Vd).

Five major metabolizing CYP450 enzymes CYP1A2, CYP2C9, CYP2C19, CYP2D6, and CYP3A4 are responsible for more than 95% of the drug metabolizing activity in humans.

The goals in evaluating in vitro drug metabolism are:

(1) to identify all of the major metabolic pathways that affect the test compound and its metabolites, including the identification of the specific enzymes responsible for metabolism and elucidation of the intermediates formed; and
(2) to explore and anticipate the effects of the test drug on the metabolism of other drugs and the effects of other drugs on its metabolism.

The most complete picture for hepatic metabolism can be obtained with intact liver systems (e.g. hepatocytes, microsomes), in which the cofactors are self-sufficient and the natural orientation and location for linked enzymes is preserved. However, when many compounds have to be tested simultaneously, a simpler screening tool is advantageous. The cDNAs for the common CYP450s have been cloned and the recombinant human enzymatic proteins have been expressed in a variety of cells. Use of these recombinant enzymes provides an excellent way to quickly assess specific enzyme inhibition activities and/or confirm results identified in microsomes.

The metabolic properties (inhibition constants on human cytochrome P450 isoforms) of the compounds described herein can be tested in vivo using the following protocol:

To assess the inhibitory activity towards CYP450 enzymes, the enzymatic reaction is monitored in the presence of different concentrations of test compound (serial dilution) and compared to maximal enzyme activity (control: no test compound). In principle, inhibition can occur by three different mechanisms: (1) competitive inhibition, (2) non-competitive inhibition, and (3) mechanism-based inhibition. In any case, the inhibition strength is dependent on the concentration of test compound. Testing the CYP450 enzyme activity over a test compound concentration range identifies the test compound concentration at which half maximal enzyme inhibition is observed (IC₅₀ concentration).

For screening purposes, the inhibitory potential of a test compound can be tested with ready to use kits (CYP450 High Throughput Inhibitor Screening kit, e.g. CYP1A2/CEC, #459500, BD Biosciences, Franklin Lakes, N.J. USA), which are available for all of the five above-mentioned major CYP isoforms. In such kits, recombinant human CYP450 isoforms expressed in insect cells are incubated with isoform specific, fluorogenic substrates in the presence of different test compound concentrations. Enzymatic activity converts the fluorogenic substrate into a fluorochrome product, the concentration of which is measured with a fluoro-spectrophotometer. Fluorescence is directly proportional to enzyme activity.

In a typical standard assay using the CYP450 High Throughput Inhibitor Screening kit, a compound is tested at 2 nM to 33 μM concentration range in a phosphate buffer (50 mM, pH 7.4) containing a glucose 6-phosphate dehydrogenase/NADP/NADPH regeneration system and a suitable fluorogenic substrate: e.g. 3-cyano-7-ethoxy-coumarin (CYP1A2). As control inhibitors, the following substances can be used: furafylline (CYP1A2), sulfaphenazole (CYP2C9), tranylcypromine (CYP2C19), quinidine (CYP2D6) and ketoconazole (CYP3A4).

The reaction is started by the addition of 2.5 nM (final concentration) CYP450 isozyme, incubated at 37° C. for 15 to 45 minutes, and then terminated by the addition of 187.5 mM tris-hydroxy-aminomethane base/acetonitrile (20/80, v/v).

The amount of generated fluorochrome is then determined by fluorescence spectroscopy with suitable exitation and emission wavelength settings: e.g. 410 nm excitation and 460 nm emission wavelength (CYP1A2).

Alternatively and/or complimentary, assays using human liver microsomes (e.g. BD Biosciences, #452161) in combination with a CYP isoform-specific standard substrate (e.g. midazolam for CYP3A4/5) as described by R. L. Walsky and R. S. Obach in Validated assay for human cytochrome p450 activities; Pharmacokinetics, Pharmacodynamics, and Drug Metabolism, Pfizer, Groton, Conn.; Drug Metabolism and Disposition: (2004)32, 647-660, can be used. To determine whether a test compound inhibits CYP3A enzyme activity, for example, hydroxylation of midazolam by human liver microsomes at varying test compound concentrations is monitored. Hydroxy-midazolam production is directly proportional to enzyme activity and can be determined by liquid chromatography-tandem mass spectrometry. Additionally, the microsomal assay can be run without and with a 15 min pre-incubation of microsomes with test compound prior to the addition of standard substrate. Test compounds or their metabolite(s) that have the potential to irreversibly modify the P450 enzyme will have a stronger inhibitory effect after pre-incubation.

In a typical standard assay using the human liver microsome assay, compounds are tested at 10 nM to 50 μM concentration range in a phosphate buffer (100 mM potassium phosphate, 3.3 mM MgCl₂, pH 7.4) containing a NADPH regeneration system (glucose 6-phosphate dehydrogenase, NADP, NADPH) and 10 μM substrate (e.g. midazolam for CYP3A4/5) and 0.1 mg/mL microsomal protein. As control inhibitors, the same substances as described above can be used (e.g. ketoconazole (CYP3A4/5)). If pre-incubation of the compound is desired, all assay components except substrate are mixed and incubated for 15 minutes at 37° C. After that period, substrate is added to the assay mix and then incubation at 37° C. is continued for 15 minutes. Without pre-incubation, all assay components are mixed simultaneously and then incubated at 37° C. for 15 minutes. Termination of the enzymatic reaction is achieved by the addition of a HCOOH/acetonitrile/H₂O (Apr. 30, 1966, v/v/v) solution. Samples are then incubated in the refrigerator (4±2° C.) for 1 h±10 min to increase protein precipitation. Directly before analysis by LC/MSMS, the samples are centrifuged at 3,500 g for 60 min at 4° C. to separate precipitated protein. The supernatant is mixed with acetonitrile/water (50/50, v/v), and then directly analyzed for compound content with LC/MSMS.

Evaluation of the data from either experimental setup is then done as follows: the fraction of remaining activity at a specific compound concentration versus the activity in the control as a function of compound concentration is used to compute IC₅₀ values. This is done by fitting a 4-parameter logistic function to the experimental data set.

The compounds of the formula (I), and preferably of the formula (IA), and their pharmaceutically acceptable salts can be used as medicines, e.g. in the form of pharmaceutical compositions. The pharmaceutical compositions can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches, ophtalmologically, e.g. in the form of solutions, suspensions, ointments, gels, pulmonary, e.g. in the form of pulmonary aerosols or to other mucosal tissues. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I), or preferably of the formula (IA), and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical products may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I), or preferably of the formula (IA), and their pharmaceutically acceptable salts in the treatment or prevention of high blood pressure, heart failure, glaucoma, myocardial infarction, renal failure, restenoses, diabetic nephropathy and stroke.

The compounds of the formula (I), and preferably of the formula (IA), and their pharmaceutically acceptable salts can also be administered in combination with one or more agents having cardiovascular activity, e.g. α- and β-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexyline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of the formulae (I) or (IA) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

The compounds of the formula (I) and their pharmaceutically acceptable salts can also be administered with one or several varying dosing intervals, as long as the intended therapeutic effect is sustained or as long as further therapeutic intervention is not required.

### EXAMPLES

The following examples illustrate the present invention. All temperatures are stated in degrees Celsius and pressures in mbar. Unless mentioned otherwise, the reactions take place at RT. The abbreviation “Rf=xx (A)” means for example that the Rf xx was found in solvent system A. The ratio of amounts of solvents to one another is always indicated in proportions by volume. Chemical names for final products and intermediates were generated with the aid of the AutoNom 2000 (Automatic Nomenclature) program, exept for spiro-compounds; whose chemical names were generated with the aid of the ACD/Name (ACD/Labs 11.0) program.

Thin-layer chromatography element systems:

A CH₂Cl₂/MeOH/NH₃ conc.=200:20:1
B CH₂Cl₂/MeOH/NH₃ conc.=200:20:0.5
C CH₂Cl₂/MeOH/NH₃ conc.=200:10:1
D CH₂Cl₂/MeOH/NH₃ conc.=90:10:1
E CH₂Cl₂/MeOH/NH₃ conc.=60:10:1
F CH₂Cl₂/MeOH/NH₃ conc.=200:30:1

### G CH₂Cl₂/MeOH=9:1

H CH₂Cl₂/MeOH/NH₃ conc.=200:15:1
I CH₂Cl₂/MeOH/NH₃ conc.=100:10:1

HPLC gradients on Hypersil BDS C-18 (5 um); column: 4×125 mm
I 90% H₂O*/10% CH₃CN* to 0% H₂O*/100% CH₃CN* in 5 min+2.5 min (1.5 ml/min)
II 95% H₂O*/5% CH₃CN* to 0% H₂O*/100% CH₃CN* in 30 min+5 min (0.8 ml/min) *contains 0.1% trifluoroacetic acid

The following abbreviations are used:
AcOH acetic acid
n-BuLi n-butyllithium
t-BuOH tert-butanol
CH₂Cl₂ dichloromethane
CHCl₃ chloroform
CH₃CN acetonitrile
Cs₂CO₃ caesium carbonate
Cy cyclohexane
DCC dicyclohexylcarbodiimide
DIBAL diisobutylaluminium hydride
DMA dimethylacetamide
4-DMAP 4-dimethylamino pyridine
DME 1,2-dimethoxyethane
DMF N,N-dimethylformamide
dppf 1,1′-bis(diphenylphosphino)-ferrocene [12150-46-8]
EDC.HCl N-ethyl-N′—(3-dimethylaminopropyl)carbodiimide hydrochloride [25952-53-8]
Et₃N triethylamine
Et₂O diethylether
EtOAc ethyl acetate
EtOH ethanol
h hour(s)
HBr hydrobromic acid
HCl hydrochloric acid
H₂O water
K₂CO₃ potassium carbonate
LiBH₄ lithium borohydride
LiCl lithium chloride
MeI methyl iodide
MeOH methanol
min minute(s)
m.p. melting point (temperature)
N₂ nitrogen
Na₂CO₃ sodium carbonate
NaH sodium hydride
NaHCO₃ sodium bicarbonate
Na₂HPO₄ di-sodium hydrogen phosphate
NaOH sodium hydroxide
Na₂SO₄ sodium sulphate
NH₃ ammonia
NH₄Br ammonium bromide
NH₄Cl ammonium chloride
NH₄OH ammonium hydroxide
Pd₂(dba)₃ tris(dibenzylideneacetone)dipalladium [51364-51-3]
Pd(PPh₃)₄ tetrakis-triphenylphosphine palladium(0)
P(tert-Bu)₃ tri-tert-butylphosphine
Ra/Ni Raney-nickel
Rf ratio of distance which a substance travels to distance of the eluent front from the start point in thin layer chromatography
Rt retention time of a substance in HPLC (in minutes)
RT room temperature
TBACl tert-butyl amminum chloride
TBAI tert-butyl amminum iodide
TBME tert-butyl methyl ether
TFA trifluoroacetic acid
THF tetrahydrofuran

### Example 1

### (1S,3′S)-6-[(2-Methoxyethoxy)methyl]-3′-{[4-(3-methoxypropyl)-3,4-dihydro-2H-1,4-benzoxazin-6-yl]methoxy}-3,4-dihydrospiro[isochromene-1,4′-piperidine]

To a solution of 1 mmol of (1S,3′S)-6-[(2-methoxyethoxy)methyl]-3′-{[4-(3-methoxy-propyl)-3,4-dihydro-2H-1,4-benzoxazin-6-yl]methoxy}-1′-[(4-methylphenyl)sulfonyl]-3,4-dihydrospiro[isochromene-1,4′-piperidine] in 6 ml of a 6:1 mixture of MeOH/THF are added 5 mmol of Na₂HPO₄ 15 mmol sodium mercury amalgam (10% Na) are added in portions and the reaction mixture is stirred at RT for 4 h (conversion checked by HPLC or TLC). The reaction mixture is diluted with CH₂Cl₂ and filtrered through a pad of silica gel. The silica gel is washed with a 2:1 mixture of CH₂Cl₂/MeOH (5×). The combined organic layers are evaporated under reduced pressure. The title compound is obtained as a slightly yellow oil from the residue by flash chromatography (SiO₂ 60 F) and is identified based on the Rf value.

The starting material(s) is (are) prepared as follows:

### a) (1S,3′S)-6-[(2-Methoxyethoxy)methyl]-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]

To solution of 1.5 mmol of 2-methoxy-ethanol [109-86-4] and 1 mmol of (1S,3′S)-6-(chloromethyl)-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine] in 6 ml of DMF are added 0.1 mmol of TBAI. The suspension is cooled to 0° C. and 1.65 mmol of NaH dispersion (60%) are added. The reaction mixture is stirred at 0° C. for 1 h and at RT for 4 h. The mixture is poured onto ice-cold H₂O and extracted with TBME (3×). The combined organic layers are washed successively with H₂O and brine, dried over Na₂SO₄ and concentrated under reduced pressure. Purification by flash chromatography (SiO₂ 60 F) affords the title compound, which is identified based on the Rf value.

### b) (1S,3′S)-6-(Chloromethyl)-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]

To a solution of 1 mmol of {(1S,3′S)-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidin]-6-yl}methanol in 5 ml of CH₂Cl₂ are added successively 1.2 mmol of Et₃N, 0.1 mmol of TBACI and 1.1 mmol of methanesulfonyl chloride at 0° C. The reaction mixture is stirred at 0° C. for 1 h and at RT for 4 h. The mixture is poured onto 1M NaHCO₃ solution and extracted with CH₂Cl₂ (2×). The cornbined organic layers are washed with brine, dried over Na₂SO₄ and concentrated under reduced pressure. Purification by flash chromatography (SiO₂ 60 F) affords the title compound as a yellow oil. Rf=0.54 (EtOAc/heptane 2:1); Rt=5.61 (gradient I).

### c) {(1S,3′S)-3′-{[4-(3-Methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidin]-6-yl}methanol

A solution of 1 mmol of (1S,3′S)-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]-6-carboxylic acid in 8 ml of THF is mixed with 3 mmol of borane-THF complex (1 M in THF) and stirred at 45° C. for 4 h (conversion checked by TLC). The reaction mixture is cooled to RT. After careful addition of 4.3 ml of MeOH, the reaction mixture is evaporated under reduced pressure. The title compound is obtained as a yellow oil from the residue by flash chromatography (SiO₂ 60 F). Rf=0.16 (EtOAc/heptane 2:1); Rt=4.78 (gradient I).

### d) (1S,3′S)-3′-{[4-(3-Methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]-6-carboxylic acid

A mixture of 1 mmol of (1S,3′S)-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]-6-carbonitrile in 5 ml of EtOH and 5 ml of 4N NaOH is heated to 80° C. for 18 h. The reaction mixture is cooled to 0° C. and 2N HCl is added until a pH of 1 is reached. The mixture is extracted with EtOAc (3×). The combined organic layers are washed H₂O and brine, dried over Na₂SO₄ and concentrated under reduced pressure. The title compound is obtained as a yellow oil. Rf=0.09 (EtOAc/heptane 2:1); Rt=4.76 (gradient I).

### e) (1S,3′S)-3′-{[4-(3-Methoxypropyl)-3,4-dihydro-2H-1,4-benzoxazin-6-yl]methoxy}-1′[(4-methylphenyl)sulfonyl]-3,4-dihydrospiro[isochromene-1,4′-piperidine]-6-carbonitrile

0.15 mmol Pd₂(dba)₃ and 0.3 mmol dppf are dissolved in 2.5 ml of DMA under argon and stirred for 10 min. Thereafter, 0.65 mmol of zinc cyanide and 1 mmol of (1S,3′S)-6-chloro-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine] in 3 ml DMA are added. The reaction mixture is stirred at 140° C. for 3 days. The mixture is cooled to RT and poured onto H₂O. The mixture is extracted with TBME (3×). The combined organic layers are washed with brine, dried over Na₂SO₄ and concentrated under reduced presssure. Purification by flash chromatography (SiO₂ 60 F) affords the title compound as a brown oil. Rf=0.22 (EtOAc/heptane 1:1); Rt=5.32 (gradient I).

### f) (1S,3′S)-6-Chloro-3′-{[4-(3-methoxypropyl)-3,4-dihydro-2H-1,4-benzoxazin-6-yl]methoxy}-1′-[(4-methylphenyl)sulfonyl]-3,4-dihydrospiro[isochromene-1,4′-piperidine]

To a solution of 1 mmol of (3S,4S)-4-[4-chloro-2-(2-hydroxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol in 12 ml of CH₂Cl₂ are added successively 3 mmol of Et₃N, 0.1 mmol of 4-DMAP and 1.5 mmol of p-toluenesulfonyl chloride at 0° C. The reaction mixture is stirred at 0° C. for 1 h and at RT for 20 h. The reaction mixture is poured onto ice/H₂O and extracted with CH₂Cl₂ (3×). The combined organic layers are dried over Na₂SO₄ and evaporated. The title compound is obtained as a slightly yellow oil from the residue by flash chromatography (SiO₂ 60 F). Rf=0.46 (EtOAc/heptane 1:1); Rt=5.86 (gradient I).

### g) (3S,4S)-[4-Chloro-2-(2-hydroxy-ethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

To a mixture of 1 mmol of (3S,4S)-4-[4-chloro-2-(2-hydroxy-ethyl)-phenyl]-3-[4-(3-methoxy-propyl)-3,4-dihydro-2H-benzo[1,4]oxazin-6-ylmethoxy]-piperidin-4-ol in 10 ml of EtOAc and 10 ml of saturated NaHCO₃ solution are added 1.05 mmol p-toluenesulfonyl chloride at 0° C. The reaction mixture is stirred for 15 h at RT. The mixture is extracted with EtOAc (3×). The combined organic layers are washed with H₂O and brine, dried over Na₂SO₄ and concentrated under reduced presssure. The title compound is obtained as a slightly yellow foam from the residue by flash chromatography (SiO₂ 60 F). Rf=0.42 (EtOAc/heptane 2:1); Rt=5.20 (gradient I).

### h) (3S,4S)-4-[4-Chloro-2-(2-hydroxy-ethyl)-phenyl]-3-[4-(3-methoxy-propyl)-3,4-dihydro-2H-benzo[1,4]oxazin-6-ylmethoxy]-piperidin-4-ol

To a solution of 1 mmol of (3S,4S)-4-[4-chloro-2-(2-hydroxy-ethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester in 2 ml of CH₂Cl₂ are added dropwise 15 mmol of TFA at 0° C. The reaction mixture is stirred at 0° C. for 30 min and at RT for 3 h (conversion checked by TLC). The reaction mixture is poured into ice-cold saturated NaHCO₃ and extracted with CH₂Cl₂ (3×). The combined organic layers are washed with H₂O, dried over Na₂SO₄ and evaporated under reduced pressure. The title compound is obtained as a slightly yellow oil. Rf=0.13 (CH₂Cl₂/MeOH/NH₄OH conc. 200:20:1); Rt=3.561 (gradient I).

### i) (3S,4S)-4-[4-Chloro-2-(2-hydroxy-ethyl)-phenyl]-3-[4-hydroxy-3-[4-(3-methoxy-propyl)-3,4-dihydro-2H-benzo[1,4]oxazin-6-ylmethoxy]-piperidine-1-carboxylic acid tert-butyl ester

To a solution of 1 mmol of (3S,4S)-4-[4-chloro-2-(2-triisopropylsilanyloxy-ethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester in 5 ml of THF are added 1.3 mmol of TBAF (1 M in THF) at RT. The mixture is stirred at RT for 2 h. The reaction mixture is poured onto ice/H₂O (100 ml) and extracted with TBME (3×). The combined organic layers are dried over Na₂SO₄ and evaporated under reduced pressure. The title compound is obtained as a yellow oil from the residue by flash chromatography (SiO₂ 60 F). Rf=0.33 (EtOAc/heptane 2:1); Rt=5.247 (gradient I).

### j) (3S,4S)-4-[4-Chloro-2-(2-triisopropylsilanyloxy-ethyl)-phenyl]-3-[4-hydroxy-3-[4-(3-methoxy-propyl)-3,4-dihydro-2H-benzo[1,4]oxazin-6-ylmethoxy]-piperidine-1-carboxylic acid tert-butyl ester

A solution of 1 mmol of (3S,4S)-4-[4-chloro-2-(2-triisopropylsilanyloxy-ethyl)-phenyl]-4-hydroxy-3-[4-(3-methoxy-propyl)-3-oxo-3,4-dihydro-2H-benzo[1,4]oxazin-6-ylmethoxy]-piperidine-1-carboxylic acid tert-butyl ester in 5 ml of THF is mixed with 2 mmol of borane-THF complex (1 M in THF) and stirred at 45° C. for 4 h (conversion checked by TLC). The reaction mixture is cooled to RT. After careful addition of 30 ml of MeOH, the reaction mixture is evaporated under reduced pressure. The title compound is obtained as yellow oil. Rf=0.62 (EtOAc/heptane 1:1).

### k) (3S,4S)-4-[4-Chloro-2-(2-triisopropylsilanyloxy-ethyl)-phenyl]-4-hydroxy-3-[4-(3-methoxy-propyl)-3-oxo-3,4-dihydro-2H-benzo[1,4]oxazin-6-ylmethoxy]-piperidine-1-carboxylic acid tert-butyl ester

To a stirred solution of 1 mmol of (3S,4S)-4-[4-chloro-2-(2-triisopropylsilanyloxy-ethyl)-phenyl]-3,4-dihydroxy-piperidine-1-carboxylic acid tert-butyl ester in 2.5 ml of DMF are added 1.1 mmol of NaH (60% dispersion in oil) at 0° C. The mixture is stirred at 0° C. for 30 min. A solution of 1.05 mmol 6-bromomethyl-4-(3-methoxy-propyl)-4H-benzo[1,4]oxazin-3-one in 1.5 ml THF is added dropwise to the reaction mixture and then 0.1 mmol of TBAI are added in one portion. The reaction mixture is stirred for 4 h at 0° C. The mixture is poured onto ice H₂O and extracted with TBME (3×). The combined organic layers are washed sucessively with H₂O and brine, dried over Na₂SO₄ and evaporated. The title compound is obtained as a yellow oil from the residue by flash chromatography (SiO₂ 60 F). Rf=0.31 (EtOAc/heptane 1:1).

### l) (3S,4S)-4-[4-Chloro-2-(2-triisopropylsilanyloxy-ethyl)-Phenul]-3,4-dihydroxy-piperidine-1-carboxylic acid tert-butyl ester

To a solution of 2 g of AD-mix-α[ALDRICH, 39, 275-8, lot 01614BE/277] in 5.5 ml of t-BuOH and 8 ml of H₂O are added 1 mmol of methanesulfonamide. The reaction mixture is cooled to 0° C. followed by the addition of 1 mmol of 4-[4-chloro-2-(2-triisopropylsilanyloxy-ethyl)-phenyl]-3,6-dihydro-2H-pyridine-1-carboxylic acid tert-butyl ester in 2.5 ml of t-BuOH. The reaction mixture is stirred at 0° C. for 30 min and then allowed to stir at RT for 10 days. Within this timeframe, four portions of AD-mix-α(0.66 g each) and methanesulfonamide (0.33 mmol each) are added to the reaction mixture. Then 3 g of Na₂SO₃ are added to the reaction mixture and stirring is continued for 1 h. The mixture is poured onto ice/H₂O and extracted with TBME (3×). The combined organic layers are washed with 2M KOH, dried over Na₂SO₄ and concentrated in vacuo. Purification by flash chromatography (SiO₂ 60 F) affords the title compound as a slightly yellow oil. Rf=0.43 (EtOAc/heptane 1:2).

### m) 4-[4-Chloro-2-(2-triisopropylsilanyloxy-ethyl)-phenyl]-3,6-dihydro-2H-pyridine-1-carboxylic acid tert-butyl ester

A three neck flask is charged with 1 mmol of 4-trifluoromethane-sulfonyloxy-3,6-dihydro-2H-pyridine-1-carboxylic acid tert-butyl ester [138647-49-1], 1.2 mmol of 4-chloro-2-(2-triisopropylsilanyloxy-ethyl)-phenyl boronic acid, 3 mmol of LiCl, 2 ml of 2N aqueous Na₂CO₃, 5 ml of DME and 0.05 mmol of Pd(PPh₃)₄. The reaction mixture is stirred for 3 h at 90° C. The reaction mixture is then cooled to RT, poured onto H₂O and extracted with TBME (3×). The combined organic layers are washed with brine, dried over Na₂SO₄ and concentrated in vacuo. Purification by flash chromatography (SiO₂ 60 F) affords the title compound as a slightly yellow oil. Rf=0.61 (EtOAc/heptane 1:3).

### n) 4-Chloro-2-(2-triisopropylsilanyloxy-ethyl)-phenyl boronic acid

A solution of 1 mmol of n-BuLi (1.6 M in hexanes) is added dropwise to a solution of 1 mmol of [2-(2-bromo-5-chloro-phenyl)-ethoxy]-triisopropyl-silane in 4 ml of THF at −78° C. The reaction mixture is stirred for 1 h at −78° C. and 2 mmol of triisopropyl borate are added during 20 min. The mixture is stirred for 30 min at −78° C. and at RT overnight. To the reaction mixture is added 0.5N HCl and the resulting mixture is extracted with EtOAc (3×). The combined organic layers are washed with brine, dried over Na₂SO₄ and concentrated in vacuo to afford the title compound as a yellow oil. Rf=0.12 (EtOAc/heptane 1:8).

### o) [2-(2-Bromo-5-chloro-phenyl)-ethoxy]-triisopropyl-silane

To a solution of 1 mmol of 2-(2-bromo-5-chloro-phenyl)-ethanol [947614-94-0] and 1.1 mmol of imidazole in 5 ml of CH₂Cl₂ are added 1.05 mmol of triisopropyl-chlorosilane at 0° C. The mixture is allowed to warm to RT and stirred for 18 h. The mixture is poured onto 0.5N HCl and extracted with CH₂Cl₂ (3×). The combined organic layers are washed with brine (1×), dried over Na₂SO₄ and concentrated in vacuo. The title compound is obtained as a yellow oil from the residue by flash chromatography (SiO₂ 60 F). Rf=0.72 (EtOAc/heptane 1:8).

According to the procedures described in example 1, the following compound(s) is(are) prepared in an analogous manner:

### 2 (1S,3′S)-6-({[(2R)-2-Ethoxypropyl]oxy}methyl)-3′-{[4-(3-methoxypropyl)-3,4-dihydro-2H-1,4-benzoxazin-6-yl]methoxy}-3,4-dihydrospiro[isochromene-1,4′-piperidine]

using (R)-2-ethoxy-propan-1-ol instead of 2-methoxy-ethanol [109-86-4] in step a Slightly yellow oil; Rf=0.21 (CH₂Cl₂/MeOH/NH₄OH conc. 200:20:1); Rt=3.91 (gradient I).

The starting material(s) is (are) prepared as follows:

### a) (R)-2-Ethoxy-propan-1-ol

To a solution of 1 mmol of (R)-2-ethoxy-propionic acid methyl ester in 3 ml of Et₂O are added 1.55 mmol LiBH₄, in portions, keeping the reaction temperature between 4-15° C. The reaction mixture is stirred at 4° C. for 1 h and 18 h at RT. The reaction mixture is poured onto saturated aqueous NH₄Cl solution over a period of 1 h keeping the temperature at 4° C. The mixture is stirred an additional 3 h at 4° C. The organic phase is separated and the aqueous phase is extracted with CH₂Cl₂ (5×). The combined organic phases are dried over Na₂SO₄ and concentrated by evaporation (35° C./200 mbar). The crude title compound is obtained as a yellow oil.

### b) (R)-2-Ethoxy-propionic acid methyl ester

To a solution of 1 mmol of methyl (R)-(+)-lactate in 5 ml of Et₂O are added 2 mmol of ethyl iodide and 2 mmol of silver oxide. The reaction mixture is stirred for 16 h at RT (conversion checked by TLC). To the reaction mixture is added 1 mmol of ethyl iodide and 1 mmol of silver oxide. The reaction mixture is stirred for 20 h at RT. The reaction mixture is filtered over Hyflo, washed with Et₂O and CH₂Cl₂ and the filtrate is concentrated by evaporation (35° C./300 mbar). Purification by flash chromatography (SiO₂ 60 F) affords the title compound as a yellow oil.

### 3 (1S,3′S)-6-({[(25)-3-Methoxy-2-methylpropyl]oxy}methyl)-3′-{[4-(3-methoxypropyl)-3,4-dihydro-2H-1,4-benzoxazin-6-yl]methoxy}-3,4-dihydrospiro[isochromene-1,4′-piperidine]

using (R)-3-methoxy-2-methyl-propan-1-ol [911855-78-2] instead of 2-methoxy-ethanol [109-86-4] in step a.

### 5 (1S,3′S)-5-[(2-Methoxyethoxy)methyl]-3′-{[4-(3-methoxypropyl)-3,4-dihydro-2H-1,4-benzoxazin-6-yl]methoxy}-3H-spiro[2-benzofuran-1,4′-piperidine]

using 4-chloro-2-(2-triisopropylsilanyloxy-methyl)-phenyl boronic acid [681128-79-0] instead of 4-chloro-2-(2-triisopropylsilanyloxy-ethyl)-phenyl boronic acid in step m.

### 6 (1S,3′S)-5-({[(2R)-2-Ethoxypropyl]oxy}methyl)-3′-{[4-(3-methoxypropyl)-3,4-dihydro-2H-1,4-benzoxazin-6-yl]methoxy}-3H-spiro[2-benzofuran-1,4′-piperidine]

using (R)-2-ethoxy-propan-1-ol (example2a) instead of 2-methoxy-ethanol [109-86-4] in step a and 4-chloro-2-(2-triisopropylsilanyloxy-methyl)-phenyl boronic acid [681128-79-0] instead of 4-chloro-2-(2-triisopropylsilanyloxy-ethyl)-phenyl boronic acid in step m.

Slightly yellow oil; Rf=0.37 (CH₂Cl₂/MeOH/NH₄OH conc. 200:20:1); Rt=4.28 (gradient I).

### 7 (1S,3′S)-5-({[(2S)-3-methoxy-2-methylpropyl]oxy}methyl)-3′-{[4-(3-methoxy-propyl)-3,4-dihydro-2H-1,4-benzoxazin-6-yl]methoxy}-3H-spiro[2-benzofuran-1,4′-piperidine]

using (R)-3-methoxy-2-methyl-propan-1-ol [911855-78-2] instead of 2-methoxy-ethanol [109-86-4] in step a. and 4-chloro-2-(2-triisopropylsilanyloxy-methyl)-phenyl boronic acid [681128-79-0] instead of 4-chloro-2-(2-triisopropylsilanyloxy-ethyl)-phenyl boronic acid in step m.

Slightly yellow oil; Rf=0.32 (CH₂Cl₂/MeOH/NH₄OH conc. 200:20:1); Rt=3.96 (gradient I).

### 9 (1S,3′S)-7-[(2-Methoxyethoxy)methyl]-3′-{[4-(3-methoxypropyl)-3,4-dihydro-2H-1,4-benzoxazin-6-yl]methoxy}-4,5-dihydro-3H-spiro[2-benzoxepine-1,4′-piperidine]

using 3-(2-bromo-5-chloro-phenyl)-propan-1-ol instead of 2-(2-bromo-5-chloro-phenyl)-ethanol [947614-94-0] in step o.

The starting material(s) is (are) prepared as follows:

### a) 3-(2-Bromo-5-chloro-phenyl)-propan-1-ol

A solution of 1 mmol of 3-(2-bromo-5-chloro-phenyl)-propionic acid [66192-05-0] in 2 ml of THF is mixed with 1.5 mmol of borane-THF complex (1 M in THF) and stirred at RT for 18 h (conversion checked by TLC). After careful addition of 80 ml of MeOH, the reaction mixture is evaporated under reduced pressure. The title compound is obtained as a yellow oil from the residue by flash chromatography (SiO₂ 60 F). Rf=0.23 (EtOAc/heptane 1:3); Rt=4.43 (gradient I).

### 10 (1S,3′S)-7-({[(2R)-2-Ethoxypropyl]oxy}methyl)-3′-{[4-(3-methoxypropyl)-3,4-dihydro-2H-1,4-benzoxazin-6-yl]methoxy}-4,5-dihydro-3H-spiro[2-benzoxepine-1,4′-piperidine]

using (R)-2-ethoxy-propan-1-ol (example2a) instead of 2-methoxy-ethanol [109-86-4] in step a and 3-(2-bromo-5-chloro-phenyl)-propan-1-ol (example 9a) instead of 2-(2-bromo-5-chloro-phenyl)-ethanol [947614-94-0] in step o.

### 11 (1S,3′S)-7-({[(2S)-3-Methoxy-2-methylpropyl]oxy}methyl)-3′-{[4-(3-methoxypropyl)-3,4-dihydro-2H-1,4-benzoxazin-6-yl]methoxy}-4,5-dihydro-3H-spiro[2-benzoxepine-1,4′-piperidine]

using (R)-3-methoxy-2-methyl-propan-1-ol [911855-78-2] instead of 2-methoxy-ethanol [109-86-4] in step a and 3-(2-bromo-5-chloro-phenyl)-propan-1-ol (example 9a) instead of 2-(2-bromo-5-chloro-phenyl)-ethanol [947614-94-0] in step o.

### Example 4

### (1S,3′S)-6-(3-Methoxypropoxy)-3′-{[4-(3-methoxypropyl)-3,4-dihydro-2H-1,4-benzoxazin-6-yl]methoxy}-3,4-dihydrospiro[isochromene-1,4′-piperidine]

To a solution of 1 mmol of tert-butyl (1S,3′S)-6-(3-methoxypropoxy)-3′-{[4-(3-methoxypropyl)-3,4-dihydro-2H-1,4-benzoxazin-6-yl]methoxy}-3,4-dihydro-1′H-spiro[isochromene-1,4′-piperidine]-1′-carboxylate in 7 ml of CH₂Cl₂ at 0° C. are added 30 mmol of TFA and the reaction mixture is stirred at 0° C. for 75 min (conversion checked by HPLC or TLC). The reaction mixture is poured into ice-cold saturated aqueous NaHCO₃ and extracted with EtOAc (2×). The combined organic layers are dried over Na₂SO₄ and evaporated. The title compound is obtained from the residue by flash chromatography (SiO₂ 60 F) and identified based on the Rf value.

The starting material(s) is (are) prepared as follows:

### a) tert-Butyl (1S,3′S)-6-(3-methoxypropoxy)-3′-{[4-(3-methoxypropyl)-3,4-dihydro-2H-1,4-benzoxazin-6-yl]methoxy}-3,4-dihydro-1′H-spiro[isochromene-1,4′-piperidine]-1′-carboxylate

To a solution of 1 mmol of (3S,4S)-4-hydroxy-4-{4-(2-methoxy-ethoxymethyl)-2-[2-(toluene-4-sulfonyloxy)-ethyl]-phenyl}-3-[4-(3-methoxy-propyl)-3,4-dihydro-2H-benzo[1,4]oxazin-6-ylmethoxy]-piperidine-1-carboxylic acid tert-butyl ester in 25 ml of DMF are added 1.2 mmol of NaH (60% dispersion in oil) at 0° C. The mixture is stirred at 0° C. for 20 min (conversion checked by LCMS). The reaction mixture is poured onto ice/H₂O and extracted with CH₂Cl₂ (2×). The combined organic layers are dried over Na₂SO₄ and evaporated. The title compound is obtained from the residue by flash chromatography (SiO₂ 60 F) and identified based on the Rf value.

### b) (3S,4S)-4-Hydroxy-4-{4-(3-methoxy-propoxy)-2-[2-(toluene-4-sulfonyloxy)-ethyl]-phenyl}-3-[4-(3-methoxy-propyl)-3,4-dihydro-2H-benzo[1,4]oxazin-6-ylmethoxy]-piperidine-1-carboxylic acid tert-butyl ester

To a solution of 1 mmol of (3S,4S)-4-hydroxy-4-[2-(2-hydroxy-ethyl)-4-(3-methoxy-propoxy)-phenyl]-3-[4-(3-methoxy-propyl)-3,4-dihydro-2H-benzo[1,4]oxazin-6-ylmethoxy]-piperidine-1-carboxylic acid tert-butyl ester in 20 ml of CH₂Cl₂ are added successively 1.5 mmol of Et₃N, 0.10 mmol of 4-DMAP and 1.2 mmol of p-toluene-sulfonyl chloride, at 0° C. The reaction mixture is stirred at 0° C. for 1 h and at RT for 60 h. The reaction mixture is poured onto ice/H₂O and extracted with CH₂Cl₂ (2×). The combined organic layers are dried over Na₂SO₄ and evaporated. The title compound is obtained from the residue by flash chromatography (SiO₂ 60 F) and identified based on the Rf value.

### c) (3S,4S)-4-Hydroxy-4-[2-(2-hydroxy-ethyl)-4-(3-methoxy-propoxy)-phenyl]-3-[4-(3-methoxy-propyl)-3,4-dihydro-2H-benzo[1,4]oxazin-6-ylmethoxy]-piperidine-1-carboxylic acid tert-butyl ester

To a solution of 1 mmol of (3S,4S)-4-hydroxy-4-[4-(3-methoxy-propoxy)-2-(2-triisopropylsilanyloxy-ethyl)-phenyl]-3-[4-(3-methoxy-propyl)-3,4-dihydro-2H-benzo[1,4]oxazin-6-ylmethoxy]-piperidine-1-carboxylic acid tert-butyl ester in 5 ml of THF are added 1.3 mmol TBAF (1 M in THF) at 0° C. The mixture is stirred at RT for 15 h. The reaction mixture is poured onto ice/H₂O and extracted with TBME (2×). The combined organic layers are dried over Na₂SO₄ and evaporated. The title compound is obtained from the residue by flash chromatography (SiO₂ 60 F) and identified based on the Rf value.

### d) (3S,4S)-4-Hydroxy-4-[4-(3-methoxy-propoxy)-2-(2-triisopropylsilanyloxy-ethyl)-phenyl]-3-[4-(3-methoxy-propyl)-3,4-dihydro-2H-benzo[1,4]oxazin-6-ylmethoxy]-piperidine-1-carboxylic acid tert-butyl ester

A solution of 1 mmol of (3S,4S)-4-hydroxy-4-[4-(3-methoxy-propoxy)-2-(2-triisopropylsilanyloxy-ethyl)-phenyl]-3-[4-(3-methoxy-propyl)-3-oxo-3,4-dihydro-2H-benzo[1,4]oxazin-6-ylmethoxy]-piperidine-1-carboxylic acid tert-butyl ester in 5 ml of THF is mixed with 3 mmol of borane-THF complex (1 M in THF) and stirred at RT for 20 h (conversion checked by LCMS). After addition of 4 ml of MeOH, the reaction mixture is evaporated. The title compound is obtained from the residue by flash chromatography (SiO₂ 60 F) and identified based on the Rf value.

### e) (3S,4S)-4-Hydroxy-4-[4-(3-methoxy-propoxy)-2-(2-triisopropylsilanyloxy-ethyl)-phenyl]-3-[4-(3-methoxy-propyl)-3-oxo-3,4-dihydro-2H-benzo[1,4]oxazin-6-ylmethoxy]-piperidine-1-carboxylic acid tert-butyl ester

To a stirred solution of 1 mmol of (3S,4S)-3,4-dihydroxy-4-[4-(3-methoxy-propoxy)-2-(2-triisopropylsilanyloxy-ethyl)-phenyl]-piperidine-1-carboxylic acid tert-butyl ester in 3.5 ml of DMF are added 1.1 mmol of NaH (60% dispersion in oil) at 0° C. The mixture is stirred at 0° C. for 30 min. Subsequently, a solution of 1.05 mmol of 6-bromomethyl-4-(3-methoxy-propyl)-4H-benzo[1,4]oxazin-3-one in 2 ml of DMF and 0.1 mmol of TBAI are added. The reaction mixture is stirred for 3 h at 0° C. The mixture is poured onto 1M aqueous NaHCO₃ and extracted with TBME (3×). The combined organic layers are washed successively with H₂O (2×) and brine, dried over Na₂SO₄ and evaporated. The title compound is obtained from the residue by flash chromatography (SiO₂ 60 F) and identified based on the Rf value.

### f) (3S,4S)-3,4-Dihydroxy-4-[4-(3-methoxy-propoxy)-2-(2-triisopropylsilanyloxy-ethyl)-phenyl]-piperidine-1-carboxylic acid tert-butyl ester

To a solution of 2 g of AD-mix-α [ALDRICH, 39, 275-8, lot 01614BE/277] in 7 ml of t-BuOH and 10 ml of H₂O are added 1 mmol of methanesulfonamide. The reaction mixture is cooled to 0° C. followed by the addition of 1 mmol of 4-[4-(3-methoxy-propoxy)-2-(2-triisopropylsilanyloxy-ethyl)-phenyl]-3,6-dihydro-2H-pyridine-1-carboxylic acid tert-butyl ester in 5 ml of t-BuOH. The reaction mixture is stirred at 0° C. for 30 min and then allowed to stir at RT for 3 days. To the reaction mixture are added 28.2 g of Na₂SO₃ followed by stirring for 1 h. The mixture is poured onto ice/H₂O and extracted with TBME (3×). The combined organic layers are washed with 2M KOH, dried over Na₂SO₄ and concentrated in vacuo. Purification by flash chromatography (SiO₂ 60 F) affords the title compound, which is identified based on the Rf value.

### g) 4[4-(3-Methoxy-propoxy)-2-(2-triisopropylsilanyloxy-ethyl)-phenyl]-3,6-dihydro-2H-pyridine-1-carboxylic acid tert-butyl ester

A three neck flask is charged with 1 mmol of 4-trifluoromethane-sulfonyloxy-3,6-dihydro-2H-pyridine-1-carboxylic acid tert-butyl ester [138647-49-1], 0.95 mmol of 4-(3-methoxy-propoxy)-2-(2-triisopropylsilanyloxy-ethyl)-phenyl-boronic acid, 3 mmol of LiCl, 2 ml of 2N aqueous Na₂CO₃, 5 ml of DME and 0.050 mmol of Pd(PPh₃)₄. The reaction mixture is stirred for 3 h at 90° C., followed by cooling to RT, poured onto water (200 ml) and extracted with TBME (3×). The combined organic layers are washed with brine, dried over Na₂SO₄ and concentrated in vacuo. Purification by flash chromatography (SiO₂ 60 F) affords the title compound, which is identified based on the Rf value.

### h) 4-(3-Methoxy-propoxy)-2-(2-triisopropylsilanyloxy-ethyl)-phenyl-boronic acid

A solution of 1.2 mmol of n-BuLi (1.6 M in hexanes) is added dropwise to the stirred solution of 1 mmol of {2-[2-bromo-5-(3-methoxy-propoxy)-phenyl]-ethoxy}-triisopropyl-silane in 10 ml of THF at −78° C. The reaction mixture is stirred for 1 h at −78° C. and 2 mmol of triisopropyl borate are added during 20 min. The mixture is stirred for 30 min at −78° C. and 1 h at RT. The reaction mixture is partitioned between 0.5N aqueous HCl and EtOAc. The aqueous phase is extracted with EtOAc (2×). The combined organic layers are washed with brine, dried over Na₂SO₄ and concentrated in vacuo to afford the title compound, which is identified based on the Rf value.

### i) {2-[2-Bromo-5-(3-methoxy-propoxy)-phenyl]-ethoxy}-triisopropyl-silane

To a solution of 1 mmol of 2-[2-bromo-5-(3-methoxy-propoxy)-phenyl]-ethanol and 1.1 mmol of imidazole in 5 ml of CH₂Cl₂ are added 1.05 mmol of triisopropylchloro-silane at 0° C. The mixture is allowed to warm to RT and stirred for 18 h. The mixture is poured onto 0.5N HCl and extracted with CH₂Cl₂ (3×). The combined organic layers are washed with brine (1×), dried over Na₂SO₄ and concentrated in vacuo. The title compound is obtained from the residue by flash chromatography and identified based on the Rf value.

### j) 2-[2-Bromo-5-(3-methoxy-propoxy)-phenyl]-ethanol

The mixture of 1 mmol of 4-bromo-3-(2-hydroxy-ethyl)-phenol [319473-28-4] in 5 ml of acteone is stirred with 2 mmol of K₂CO₃ and 1.1 mmol of 1-bromo-3-methoxy-propane [36865-41-5] at reflux temperature over 22 h. The mixture is poured onto ice/H₂O and extracted with TBME (2×). The combined organic layers are washed with brine, dried over Na₂SO₄ and concentrated in vacuo. Purification by flash chromatography (SiO₂ 60 F) affords the title compound, which is identified based on the Rf value.

According to the procedures described in example 4, the following compound(s) is(are) prepared in an analogous manner:

### 8 (1S,3′S)-5-(3-Methoxypropoxy)-3′-{[4-(3-methoxypropyl)-3,4-dihydro-2H-1,4-benzoxazin-6-yl]methoxy}-3H-spiro[2-benzofuran-1,4′-piperidine]

using 4-bromo-3-hydroxymethyl-phenol [2737-20-4] instead of 4-bromo-3-(2-hydroxy-ethyl)-phenol [319473-28-4] in step j.

### 12 (1S,3′S)-7-(3-Methoxypropoxy)-3′-{[4-(3-methoxypropyl)-3,4-dihydro-2H-1,4-benzoxazin-6-yl]methoxy}-4,5-dihydro-3H-spiro[2-benzoxepine-1,4′-piperidine]

using 4-bromo-3-(3-hydroxy-propyl)-phenol instead of 4-bromo-3-(2-hydroxy-ethyl)-phenol [319473-28-4] in step j.

The starting material(s) is (are) prepared as follows:

### a) 4-Bromo-3-(3-hydroxy-propyl)-phenol

A solution of 1 mmol of 3-(2-bromo-5-hydroxy-phenyl)-propionic acid methyl ester [936758-64-4] in 8 ml of THF is mixed with 2 mmol of LiAlH₄ (1M in THF) and stirred at RT for 13 h (conversion checked by HPLC or TLC), then the reaction mixture is poured on saturated aqueous NaHCO₃ solution and extracted with TBME (3×). The combined organic phases are washed with H₂O and brine and evaporated in vacuo. The title compound is obtained from the residue by flash chromatography (SiO₂ 60 F) and identified based on the Rf value.

### 13 (3′S,5S)-8-[(2-Methoxyethoxy)methyl]-3′-{[4-(3-methoxypropyl)-3,4-dihydro-2H-1,4-benzoxazin-6-yl]methoxy}-2,3-dihydrospiro[1,4-benzodioxepine-5,4′-piperidine]

using {2-[2-bromo-5-(2-methoxy-ethoxymethyl)-phenoxy]-ethoxy}-triisopropyl-silane instead of {2-[2-bromo-5-(3-methoxy-propoxy)-phenyl]-ethoxy}-triisopropyl-silane (example 4i) in step h.

The starting material(s) is (are) prepared as follows:

### a) {2-[2-Bromo-5-(2-methoxy-ethoxymethyl)-phenoxy]-ethoxy}-triisopropyl-silane

The solution of 1.3 mmol of 2-methoxy-ethanol [109-86-4], 1 mmol of [2-(2-bromo-5-chloromethyl-phenoxy)-ethoxy]-triisopropyl-silane in 5 ml DMF is admixed with stirring at −10° C. with 1.2 mmol of NaH dispersion (60%) and 0.1 mmol of TBAI. The reaction mixture is stirred at −10° C. for 1 h and at RT for 18 h. The mixture is poured onto 1M aqueous NaHCO₃ solution and extracted with TBME (3×). The organic phases are washed successively with H₂O (2×) and brine, dried over Na₂SO₄ and concentrated by evaporation. Purification by flash chromatography (SiO₂ 60 F) affords the title compound, which is identified based on the Rf value.

### b) [2-(2-Bromo-5-chloromethyl-phenoxy)-ethoxy]-triisopropyl-silane

To a solution of 1 mmol of [4-bromo-3-(2-triisopropylsilanyloxy-ethoxy)-phenyl]-methanol in 5 ml of CH₂Cl₂ are added successively 1.2 mmol of Et₃N, 0.1 mmol of TBAI and 1.1 mmol of methanesulfonyl chloride at 0° C. The reaction mixture is stirred at 0° C. for 1 h and at RT for 20 h. The mixture is poured onto 1M aqueous NaHCO₃ solution and extracted with CH₂Cl₂ (2×). The organic phases are washed with brine, dried over Na₂SO₄ and concentrated by evaporation. Purification by flash chromatography (SiO₂ 60 F) affords the title compound as a slightly yellow oil. Rf=0.64 (EtOAc/heptane 1:4); Rt=7.07 (gradient I).

### c) [4-Bromo-3-(2-triisopropylsilanyloxy-ethoxy)-phenyl]-methanol

To a solution of 1 mmol of 4-bromo-3-(2-triisopropylsilanyloxy-ethoxy)-benzoic acid methyl ester in 15 ml of THF at RT are added 3 mmol LiBH₄. The reaction mixture is stirred at 50° C. for 24 h. The cooled reaction mixture is poured onto 1 M aqueous NH₄Cl solution and extracted with TBME (2×). The combined organic phases are washed with brine, dried over Na₂SO₄ and concentrated by evaporation. Purification by crystallization (from heptane) affords the title compound as white crystals.

Rf=0.11 (EtOAc/heptane 1:4); Rt=6.43 (gradient I). Mp 62.2° C.

### d) 4-Bromo-3-(2-triisopropylsilanyloxy-ethoxy)-benzoic acid methyl ester

The mixture of 1 mmol of 4-bromo-3-hydroxy-benzoic acid methyl ester [106291-80-9] in 5 ml of acteone is stirred with 2 mmol of K₂CO₃ and 1.1 mmol of (2-iodo-ethoxy)-triisopropyl-silane [93550-77-7] at reflux temperature over 22 h. The mixture is poured onto ice/H₂O and extracted with TBME (2×). The combined organic layers are washed with brine, dried over Na₂SO₄ and concentrated in vacuo. Purification by flash chromatography (SiO₂ 60 F) affords the title compound after crystallization (from heptane) as white crystals. Rf=0.15 (EtOAc/heptane 1:4); Rt=7.14 (gradient I).

### 14 (3′S,5S)-8-({[(2R)-2-Ethoxypropyl]oxy}methyl)-3′-{[4-(3-methoxypropyl)-3,4-dihydro-2H-1,4-benzoxazin-6-yl]methoxy}-2,3-dihydrospiro[1,4-benzo-dioxepine-5,4′-piperidine]

using {2-[2-Bromo-5-((R)-2-ethoxy-propoxymethyl)-phenoxy]-ethoxy}-triisopropyl-silane instead of {2-[2-bromo-5-(3-methoxy-propoxy)-phenyl]-ethoxy}-triisopropyl-silane (example 4i) in step h.

Slightly yellow oil. Rf=0.30 (CH₂Cl₂/MeOH/NH₃ conc. 80:10:1); Rt=3.95 (gradient I).

The starting material(s) is (are) prepared as follows:

### i) {2-[2-Bromo-5-((R)-2-ethoxy-propoxymethyl)-phenoxy]-ethoxy}-triisopropyl-silane

The starting material is obtained according to the procedure described in example 13a using (R)-2-ethoxy-propan-1-ol (example 2a) instead of 2-methoxy-ethanol [109-86-4]. Slightly yellow oil. Rf=0.45 (EtOAc/heptane 1:4); Rt=7.52 (gradient I).

### 15 (3′S,5S)-8-({[(2S)-3-Methoxy-2-methylpropyl]oxy}methyl)-3′-{[4-(3-methoxypropyl)-3,4-dihydro-2H-1,4-benzoxazin-6-yl]methoxy}-2,3-dihydrospiro[1,4-benzodioxepine-5,4′-piperidine]

using {2-[2-bromo-5-((S)-3-methoxy-2-methyl-propoxymethyl)-phenoxy]-ethoxy}-triisopropyl-silane instead of {2-[2-bromo-5-(3-methoxy-propoxy)-phenyl]-ethoxy}-triisopropyl-silane (example 4i) in step h.

The starting material(s) is (are) prepared as follows:

### a) {2[2-Bromo-5-((S)-3-methoxy-2-methyl-propoxymethyl)-phenoxy]-ethoxy}-triisopropyl-silane

The starting material is obtained according to the procedure described in example 13a using (R)-3-methoxy-2-methyl-propan-1-ol [911855-78-2] instead of 2-methoxy-ethanol [109-86-4]. Slightly yellow oil. Rf=0.45 (EtOAc/heptane 1:4); Rt=7.52 (gradient I).

### 16 (3′S,55)-8-(3-Methoxypropoxy)-3′-{[4-(3-methoxypropyl)-3,4-dihydro-2H-1,4-benzoxazin-6-yl]methoxy}-2,3-dihydrospiro[1,4-benzodioxepine-5,4′-piperidine] using {2-[2-bromo-5-(3-methoxy-propoxy)-phenoxy]-ethoxy}-triisopropyl-silane instead of {2-[2-bromo-5-(3-methoxy-propoxy)-phenyl]-ethoxy}-triisopropyl-silane (example 4i) in step h.

The starting material(s) is (are) prepared as follows:

### a) {2[2-Bromo-5-(3-methoxy-propoxy)-phenoxy]-ethoxy}-triisopropyl-silane

A mixture of 1 mmol of 4-bromo-benzene-1,3-diol [6626-15-9], 6.5 mmol of K₂CO₃, and 15 ml of dry acetone is stirred at RT for 30 min. To the mixture is added 1 mmol of 1-bromo-3-methoxy-propane [36865-41-5] and the mixture is heated to reflux. After 23 h, 2.7 mmol of (2-iodo-ethoxy)-triisopropyl-silane [93550-77-7] are added and the mixture is refluxed again for 28 h. After cooling, the mixture is filtered through a kieselguhr plug and the filtrate is evaporated. The residue is purified by flash chromatography (SiO₂ 60 F) to afford the title compound, which is identified based on the Rf value.

1. A compound of the general formula 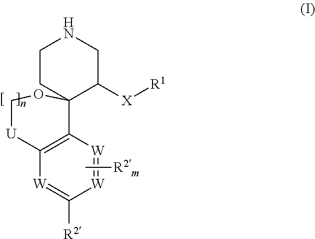
its prodrug, its nitrate-ester or nitrosated derivative or its pharmaceutically acceptable salt, in which
R¹ is aryl or heterocyclyl, each of which is substituted by 1-4 radicals independently selected from the group consisting of
acyl-C₁₋₈-alkoxy-C₁₋₈-alkoxy,
acyl-C₁₋₈-alkoxy-C₁₋₈-alkyl,
(N-acyl)-C₁₋₈-alkoxy-C₁₋₈-alkylamino,
C₁₋₈-alkanoyl,
C₁₋₈-alkoxy,
C₁₋₈-alkoxy-C₁₋₈-alkanoyl,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
(N—C₁₋₈-alkoxy)-C₁₋₈-alkylaminocarbonyl-C₁₋₈-alkoxy,
(N—C₁₋₈-alkoxy)-C₁₋₈-alkylaminocarbonyl-C₁₋₈-alkyl,
C₁₋₈-alkoxy-C₁₋₈-alkylcarbamoyl,
C₁₋₈-alkoxy-C₁₋₈-alkylcarbonyl,
C₁₋₈-alkoxy-C₁₋₈-alkylcarbonylamino,
C₁₋₈-alkoxycarbonyl,
C₁₋₈-alkoxycarbonyl-C₁₋₈-alkoxy,
C₁₋₈-alkoxycarbonyl-C₁₋₈-alkyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₁₋₈-alkoxy-C₁₋₈-alkylcarbamoyl,
(N—C₁₋₈-alkyl)-C₁₋₈-alkoxy-C₁₋₈-alkylcarbonylamino,
(N—C₁₋₈-alkyl)-C₁₋₈-alkoxycarbonylamino,
(N—C₁₋₈-alkyl)-C₁₋₈-alkylcarbonylamino-C₁₋₈-alkoxy,
(N—C₁₋₈-alkyl)-C₁₋₈-alkylcarbonylamino-C₁₋₈-alkyl,
(N—C₁₋₈-alkyl)-C₁₋₈-alkylsulfonylamino-C₁₋₈-alkoxy,
(N—C₁₋₈-alkyl)-C₁₋₈-alkylsulfonylamino-C₁₋₈-alkyl,
C₁₋₈-alkylamidinyl,
C₁₋₈-alkylamino-C₁₋₈-alkoxy,
di-C₁₋₈-alkylamino-C₁₋₈-alkoxy,
C₁₋₈-alkylamino-C₁₋₈-alkyl,
di-C₁₋₈-alkylamino-C₁₋₈-alkyl,
C₁₋₈-alkylaminocarbonyl-C₁₋₈-alkoxy,
di-C₁₋₈-alkylaminocarbonyl-C₁₋₈-alkoxy,
C₁₋₈-alkylaminocarbonyl-C₁₋₈-alkoxy-C₁₋₈-alkyl,
C₁₋₈-alkylaminocarbonyl-C₁₋₈-alkyl,
di-C₁₋₈-alkylaminocarbonyl-C₁₋₈-alkyl,
C₁₋₈-alkylaminocarbonylamino-C₁₋₈-alkoxy,
C₁₋₈-alkylaminocarbonylamino-C₁₋₈-alkyl,
C₀₋₈-alkylcarbonylamino,
C₀₋₈-alkylcarbonylamino-C₁₋₈-alkoxy,
C₀₋₈-alkylcarbonylamino-C₁₋₈-alkyl,
C₁₋₈-alkylcarbonyloxy-C₁₋₈-alkoxy,
C₁₋₈-alkylcarbonyloxy-C₁₋₈-alkyl,
C₁₋₈-alkylsulfonyl,
C₁₋₈-alkylsulfonyl-C₁₋₈-alkoxy,
C₁₋₈-alkylsulfonyl-C₁₋₈-alkyl,
C₁₋₈-alkylsulfonylamino-C₁₋₈-alkoxy,
C₁₋₈-alkylsulfonylamino-C₁₋₈-alkyl,
optionally N-mono- or N,N-di-C₁₋₈-alkylated amino,
unsubstituted or substituted aryl-C₀₋₈-alkoxy,
unsubstituted or substituted aryl-C₀₋₈-alkyl, preferably halogen substituted-aryl,
optionally N-mono- or N,N-di-C₁₋₈-alkylated carbamoyl-C₀₋₈-alkoxy,
optionally N-mono- or N,N-di-C₁₋₈-alkylated carbamoyl-C₀₋₈-alkyl,
carboxy-C₁₋₈-alkoxy,
carboxy-C₁₋₈-alkoxy-C₁₋₈-alkyl,
carboxy-C₁₋₈-alkyl,
cyano,
cyano-C₁₋₈-alkoxy,
cyano-C₁₋₈-alkyl,
unsubstituted or substituted C₃₋₁₂-cycloalkyl-C₁₋₈-alkoxy,
unsubstituted or substituted C₃₋₁₂-cycloalkyl-C₁₋₈-alkyl,
unsubstituted or substituted C₃₋₁₂-cycloalkylcarbonylamino-C₁₋₈-alkoxy,
unsubstituted or substituted C₃₋₁₂-cycloalkylcarbonylamino-C₁₋₈-alkyl,
O,N-dimethylhydroxylamino-C₁₋₈-alkyl,
halogen,
halogen substituted C₁₋₈-alkoxy,
halogen substituted C₁₋₈-alkyl,
unsubstituted or substituted heterocyclyl-C₀₋₈-alkoxy,
unsubstituted or substituted heterocyclyl-C₀₋₈-alkyl, preferably C₁₋₈-alkoxy-C₁₋₈-alkylheterocyclyl,
unsubstituted or substituted heterocyclylcarbonyl,
hydroxy-C₁₋₈-alkoxy-C₁₋₈-alkoxy,
hydroxy-C₁₋₈-alkoxy-C₁₋₈-alkyl,
hydroxy-C₁₋₈-alkyl,
O-methyloximyl-C₁₋₈-alkyl,
oxide and oxo;
where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;
R^{2′} is independently selected from the group consisting of
C₁₋₈-alkanoyloxy-C₁₋₈-alkyl,
C₂₋₈-alkenyl,
C₂₋₈-alkenyloxy,
C₂₋₈-alkenyloxy-C₁₋₈-alkyl,
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
C₁₋₈-alkoxy-C₁₋₈-alkyl,
C₁₋₈-alkoxy-C₁₋₈-alkylamino-C₁₋₈-alkyl,
C₁₋₈-alkoxy-C₁₋₈-alkylsulfanyl,
C₁₋₈-alkoxy-C₁₋₈-alkylsulfanyl-C₁₋₈-alkyl,
C₁₋₈-alkoxycarbonyl,
C₁₋₈-alkoxycarbonyloxy-C₁₋₈-alkyl,
C₁₋₈-alkoxy-C₃₋₈-cycloalkyl-C₁₋₈-alkyl,
C₁₋₈-alkyl,
C₁₋₈-alkylsulfanyl,
C₁₋₈-alkylsulfanyl-C₁₋₈-alkoxy,
C₁₋₈-alkylsulfanyl-C₁₋₈-alkoxy-C₁₋₈-alkyl,
C₁₋₈-alkylsulfanyl-C₁₋₈-alkyl,
C₁₋₈-alkylsulfonyl-C₁₋₈-alkoxy-C₁₋₈-alkyl,
C₁₋₈-alkylsulfonyl-C₁₋₈-alkyl,
C₂₋₈-alkynyl,
optionally substituted C₁₋₈-alkoxy,
optionally N-mono- or N,N-di-C₁₋₈-alkylated amino-C₁₋₈-alkoxy,
optionally N-mono- or N,N-di-C₁₋₈-alkylated amino-carbonyl-C₁₋₈-alkyl,
unsubstituted or substituted aryl-C₁₋₈-alkoxy-C₁₋₈-alkoxy,
unsubstituted or substituted aryl-heterocyclyl-C₀₋₈-alkoxy,
unsubstituted or substituted heterocyclyl-heterocyclyl-C₀₋₈-alkoxy,
unsubstituted or substituted aryloxy,
unsubstituted or substituted aryl-C₀₋₈-alkoxy-C₁₋₈-alkoxy,
unsubstituted or substituted aryl-C₀₋₈-alkoxy-C₁₋₈-alkoxy-C₁₋₈-alkyl,
carboxy-C₁₋₈-alkyl,
cyano,
cyano-C₁₋₈-alkyl,
unsubstituted or substituted C₃₋₈-cycloalkyl-C₀₋₈-alkoxy-C₇₋₈-alkoxy,
unsubstituted or substituted C₃₋₈-cycloalkyl-C₀₋₈-alkoxy-C₁₋₈-alkoxy-C₁₋₈-alkyl,
unsubstituted or substituted C₃₋₈-cycloalkyl-C₀₋₈-alkoxy-C₁₋₈-alkyl, preferably C₁₋₈-alkoxy-C₀₋₈-alkyl-C₃₋₈-cycloalkyl-C₀₋₈-alkoxy-C₁₋₈-alkyl,
unsubstituted or substituted C₃₋₈-cycloalkyl-C₀₋₈-alkylamino-C₁₋₈-alkyl,
halogen-substituted C₁₋₈-alkoxy,
halogen-substituted C₁₋₈-alkyl,
halogen-substituted C₁₋₈-alkoxy-C₁₋₈-alkoxy-C₁₋₈-alkyl,
unsubstituted or substituted heterocyclyl-carbonyl-C₁₋₈-alkyl,
unsubstituted or substituted heterocyclyl-C₁₋₈-alkyl,
unsubstituted or substituted heterocyclyl-sulfanyl-C₁₋₈-alkoxy-C₁₋₈-alkyl,
unsubstituted or substituted heterocyclyl-C₀₋₈-alkoxy-C₁₋₈-alkoxy and
unsubstituted or substituted heterocyclyl-C₀₋₈-alkoxy-C₁₋₈-alkyl;
X is -Alk-, —O-Alk-, -Alk-O—, —O-Alk-O—, —S-Alk-, -Alk-S—, -Alk-NR⁴—, —NR⁴-Alk-, —C(O)—NR⁴—, -Alk-C(O)—NR⁴—, -Alk-C(O)—NR⁴—, Alk-, —NR⁴—C(O)—, -Alk-NR⁴—C(O)—, —NR⁴—C(O)-Alk-, -Alk-NR⁴—C(O)-Alk-, —O-Alk-C(O)—NR⁴—, —O-Alk-NR⁴—C(O)—, —S(O)₂—NR⁴— or —S(O)₂—NR⁴-Alk-, where Alk is C₁₋₈-alkylene which may optionally be substituted by halogen;
R⁴ is hydrogen, C₁₋₈-alkyl, C₁₋₈-alkoxy-C₁₋₈-alkyl, acyl, unsubstituted or substituted C₃₋₈-cycloalkyl or unsubstituted or substituted aryl-C₁₋₈-alkyl;
U is selected from the group consisting of —CH₂—, NR⁴, —O— and S(O)ₚ;
W is independently selected from the group consisting of —CH═ and —N═, whereby a maximum of one W can be —N═;
n is 0-2 if U is —CH₂— or n is 2 if U is —O—, NR^{4,} or S(O)ₚ;
m is 0-3 if all W are —CH═; or m is 0-2, if one W is —N═; and
p is 0-2.

2. A compound according to claim 1, which corresponds to the general formula (IA) 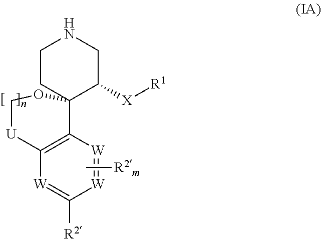
its prodrug, its nitrate-ester or nitrosated derivative or its pharmaceutically acceptable salt, where the meanings of the substituents R¹, R^{2′}, X, U, W, m and n are as indicated for compounds of the formula (I) according to claim 1.

3. A compound according to claim 1 or 2, wherein
R¹ is
2H-chromenyl,
3,4-dihydro-2H-benzo[1,4]oxazinyl,
3,4-dihydro-2H-benzo[1,4]thiazinyl or
1,3-dihydroindolyl
substituted by 1-3 radicals independently selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
C₁₋₈-alkoxy-C₁₋₈-alkylcarbonyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₀₋₈-alkylcarbonylamino-C₁₋₈-alkoxy,
(N—C₁₋₈-alkyl)-C₀₋₈-alkylcarbonylamino-C₁₋₈-alkyl,
C₀₋₈-alkylcarbonylamino-C₁₋₈-alkoxy,
C₀₋₈-alkylcarbonylamino-C₁₋₈-alkyl,
halogen,
oxo,
halogen-substituted C₁₋₈-alkoxy and
halogen-substituted C₁₋₈-alkyl, or a pharmaceutically acceptable salt thereof.

4. A compound according to any one of claims 1 to 3, wherein
R^{2′} is selected from the group consisting of
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkyl,
optionally substituted C₁₋₈-alkoxy,
C₁₋₈-alkyl,
unsubstituted or substituted C₃₋₈-cycloalkyl-C₀₋₈-alkoxy-C₁₋₈-alkyl,
unsubstituted or substituted heterocyclyl-C₀₋₈-alkoxy-C₁₋₈-alkyl and
unsubstituted or substituted optionally substituted heterocyclyl-pyrrolidinyl-C₀₋₈-alkoxy, or a pharmaceutically acceptable salt thereof.

5. A compound according to any one of claim 1, 2 or 4, wherein
R¹ is 2H-chromenyl or 3,4-dihydro-2H-benzo[1,4]oxazinyl, substituted as defined for a compound of formula (I) according to claim 1;
R^{2′} is selected from the group consisting of
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkyl,
optionally substituted C₁₋₈-alkoxy,
C₁₋₈-alkyl,
unsubstituted or substituted C₃₋₈-cycloalkyl-C₀₋₈-alkoxy-C₁₋₈-alkyl,
unsubstituted or substituted heterocyclyl-C₀₋₈-alkoxy-C₁₋₈-alkyl and
unsubstituted or substituted optionally substituted heterocyclyl-pyrrolidinyl-C₀₋₈-alkoxy;
X is -Alk-, —O-Alk- or —O-Alk-O— where Alk is C₁₋₈-alkylene;
U is selected from the group consisting of —CH₂— and —O—;
W is in each case —CH═;
n is 0-2 if U is —CH₂— or n is 2 if U is —O—; and
m is 0, or a pharmaceutically acceptable salt thereof.

6-8. (canceled)

9. A method for preventing, for delaying the progression of or for treating high blood pressure, heart failure, glaucoma, myocardial infarction, renal failure, restenoses, diabetic nephropathy or stroke, where a therapeutically effective amount of a compound of the general formula (I) or (IA) or a pharmaceutically acceptable salt thereof, according to any one of claims 1 to 5, is used.

10. A pharmaceutical product comprising a compound of the general formula (I) or (IA) or a pharmaceutically acceptable salt thereof, according to any one of claims 1 to 5, and conventional excipients.

11. A pharmaceutical combination in the form of a product or of a kit composed of individual components consisting a) of a compound of the general formula (I) or (IA) or a pharmaceutically acceptable salt thereof, according to claim 1, and b) at least one pharmaceutical form as active ingredient having a cardiovascular effect.
